(19) United States
(12) Patent Application Publication
(10) Pub. No.: US 2013/0118485 A1
(43) Pub. Date: May 16, 2013

(54) MEANS AND METHOD FOR ADMINISTERING MEDICAMENTS TO INFANTS

(71) Applicant: Daniel Shahaf, M.P. Emek Ha-Yarden (IL)

(72) Inventor: Daniel Shahaf, M.P. Emek Ha-Yarden (IL)

(73) Assignee: KAST SILICONE LTD., Kiryat Anavim (IL)

(21) Appl. No.: 13/639,317

(22) PCT Filed: Apr. 7, 2011
(86) PCT No.: PCT/IL11/00293

§ 371 Date: Dec. 21, 2012

Related U.S. Application Data
(60) Provisional application No. 61/321,505, filed on Apr. 7, 2010.

(51) Int. Cl. A61J 17/00; A61M 16/01; A61M 16/06

(52) U.S. Cl. CPC A61J 17/00; A61M 16/06; A61M 16/01
USPC 128/202.15; 128/202.16

(57) ABSTRACT

An infant mouth-nose mask for administering medicaments to infants, comprising: a. a mouth portion, defining a confined mouth volume, placed over the mouth of said infant characterized by having at least one perforation reversibly coupled to calming or feeding means such that said infant remains relaxed and soothed while sucking said calming or feeding means; said confined mouth volume is characterized by volume, Vₘₒᵤₜₕ; and, b. a nose portion, defining a confined mouth volume, placed over the nose of said infant; said nose portion is adapted to be in fluid connection with a medicament delivery source (MDS); said confined nose volume is characterized by volume, Vₙₒₛₑ; wherein said Vₙₒₛₑ>>a*Vₘₒᵤₜₕ such that the dead-space of said mask is not greater than 28 cc, and a is in the range of about 1.5 to about 2.

### FIELD OF THE INVENTION

The present invention relates generally to—improved aerosolized drug administering devices, preferably for use by children and infants.

### BACKGROUND OF THE INVENTION

A significant problem faced by the pharmaceutical industry is the need for effective devices for the delivery of drugs to infants and children, in particular drugs in the form of aerosols, powders or gases which are administered by inhalation. It has been a known fact that while infants or children are administered aerosol medicament, the existing devices usually make them cry so that they reject—the device and the treatments and thus virtually none of the medicament gets to the lungs.

By contrast, if the child is breathing quietly and comfortably, the medicament will be delivered to the lungs quite efficiently.

Moreover, small children and infants typically find standard inhalation devices frightening and, as a consequence, reject them. Faced with considerable resistance from most children, many care-givers responsible for administering medication to them report a reluctance to offer aerosol medicament—for use with standard inhalation devices on a regular basis. In addition, care-givers also report that even when attempted, the delivery of aerosol-medication to children is often poor because the child cries, squirms and/or forcibly pushes the mask away from their face before adequate inhalation of the medication is complete.

Inhalation devices have been described which employ some form of auditory or visual warning or feedback notification of the passage of inhaled air containing the aerosol medicament to ensure that the pharmaceutical therapeutic agent ejected from the drug delivery means actually reaches the patient. For example, U.S. Pat. No. 4,984,158; Australian Patent No. 620375; Australian Patent No. 618789; U.S. Pat. Nos. 5,042,467; 5,363,842; 5,431,154; 5,522,380; United Kingdom Patent Application No. 2 299 512A; U.S. Pat. No. 5,758,638; French Patent Application No. 2 763 507A, and International Patent Publication No. WO 94/44974 all describe—such inhalation devices.

However, inhalation devices which merely provide for an auditory or visual signal to monitor a correct breathing pattern and/or drug delivery do not generally utilize a method that is both capable of providing feedback to medical personnel and/or other caregivers to allow them to determine whether the medicament has been adequately inhaled, while simultaneously providing means of delivering aerosol medications that is sufficiently pleasant to provide an inducement for their correct use by infants and small children. Accordingly, such devices merely provide feedback to caregivers without addressing the significant problem of fear and rejection which infants and small children have of facemasks required in order to deliver aerosol medicaments to these infants and children.

Sucking is a natural and frequent act of infants and—is associated with great comfort and has a calming or soothing action on infants.

Therefore, devices that will inherently couple the soothing effect with—improved and ensured administration of the drug would be superior—to current techniques.

Examples of a device that incorporates soothing means in order to overcome the infant's rejection to and from the device and the treatment are the following

U.S. Pat. No. 7,134,432 ('432) to Thomas Olsen. Patent '432 relates to a breathing aid device for infant which is adapted for delivery of inhalable medication in a gentle flow to an area immediately adjacent the baby's nasal passageway. An oral member formed as a pacifier carries a hollow tube, the open top end of which extends just above the upper rim of the mouth guard of the pacifier. A swivel connector secured to the bottom of the tube allows easy adjustment of the position of the breathing aid in relation to the mouth guard and to a connecting hose, through which oxygen or other medication is delivered.

However, patent '432 uses a nebulizer only, while the present invention relates to the use of pMDIs and valved aerosol holding chambers or reservoirs and also with nebulizer.

Another profound issue encountered with conventional inhalation devices is the fact that the medicament effective dose inhaled by the infant is usually negligible. One reason for that might be the accumulation of the medicament within the device so that it is not available for inhalation by the patient (for example if the medicament remains within the device for too long because of difficulty getting the child to accept the mask and/or is attracted to the device walls by static charge).

Whereas, patent '432 does disclose the use of the full coordination between sucking the soother and breathing the medicament, nor does it refer—to the negligible amount of medicament that is likely to be inhaled by the infant.

A further issue which is not disclosed in '432 is the fact that a non neglectable amount of medicament reaches the infant's eyes region. This issue is highly important and is not refereed to in '432. On the contrary, the present invention provides means and methods especially for resulting said issue.

Another example of such teaching is U.S. Pat. No. 6,470,882 ('882) to Michael Newhouse and Israel Amirav. Unlike patent '432, patent '882 does describe the use of a pMDI and spacer or valved aerosol holding chamber (reservoir). However, it does not refer to the accumulation of medicament on the walls of the reservoir nor to the negligible amount of medicament that is actually delivered to the infant.

Furthermore, patent '882 claims a device that provides “substantially sealed space” between the mask and the infant's face. It is acknowledged by the present invention that for optimum therapy, the edge of the mask must create a sealed environment with the infant's face during treatment. It has been shown that even a 1 cm gap between the mask and the face reduces the dose delivered by 50% (Everard M L, Clark A R, Milner A D; Drug delivery from jet nebulisers; Arch Dis Child 1992; 67: 586-91). Achieving a good mask-face seal—may be difficult in many infants because of squirming and crying (Amirav I, Newhouse M T; Aerosol therapy with valved holding chambers in young children: importance of the facemask seal; Pediatrics 2001; 108: 389-94). It is acknowledged in the literature that the likelihood of poor aerosol delivery to infants using face masks, is primarily because of an inadequate face mask seal (see (a) Amirav I, Newhouse M T. Aerosol therapy with valved holding chambers in young children: importance of the facemask seal; Pediatrics 2001; 108: 389-94; (b) Janssens H M, Devadason S G, Hop W C, et al. Variability of aerosol delivery via spacer devices in young asthmatic children in daily life; Eur Respir J 1999; 13: 787-91). Persisting with a screaming infant as parents often do, is not a good solution as it has been shown that little aerosol medication is deposited in the lungs (see (a) Tal A, Golan H, Grauer N, et al. Deposition pattern of radiolabeled salbutamol inhaled from a metered-dose inhaler by means of a valved holding chamber/spacer reservoir with mask in young children with airway obstruction. J Pediatr 1996; 128; 479-84; (b) Murakami G, Igarashi T, Adachi Y, et al. Measurement of bronchial hyperreactivity in infants and preschool children using a new method; Ann Allergy 1990; 64: 383-7) and using force may make subsequent aerosol treatments even more difficult. Therefore it would be very advantageous to provide an inherently 80-100% sealed space adjacent to the child's nose without the need for the caregiver to apply any external force.

Patent '882 discloses in the detail description (column 5, lines 5-10) the following “As such, in contrast to current devices, the invention will allow the baby to almost certainly tolerate the 20 seconds of drug administration (5-6 breaths) and, therefore, is more likely to improve asthma control or provide relief from an asthma attack”, thus such a device could not be used to provide the user a therapy use requires a prolong treatment (more than 20 seconds).

Furthermore, patent '882 is directed to provide a method of administering medicament to infants directly to the nose of the infant so as to increase the efficiency of the treatment: “Most babies and infants are obligatory nose breathers most of the time (with the exception of infants with nasal obstruction due to the common cold, etc., or while crying) and it is therefore more logical to emphasize the nasal route for inhalation when devising an MDI accessory aerosol delivery system. Indeed, even when a face mask is used, the aerosol is actually inhaled most of the time through the nose. The face mask thus has a much larger dead space than necessary, which in situations of low tidal volume such as in neonates or infants can considerably reduce the efficiency of delivery of aerosol medication. A small mask that preferentially directs the aerosol towards the infant's nose is thus superior to a face mask. Furthermore, aerosol delivered by means of a face mask must pass across the lower half of the face to get to the nostrils. Much of the steroid is thus actually delivered to the skin of the face, and there have been case reports of steroid side effects such as acne under these conditions. By using a nasal mask or a system that directs the aerosol towards the nose, this problem would be minimized or eliminated since the aerosol would pass directly into the nose and from there into the lungs” (see column 2, lines 18-39); “It is the principal object of the present invention to utilize a nasal mask with an existing aerochamber (see column 2, lines 66-67); “The nasal mask directs the aerosol of medication droplets or dry particles to the nose and thus does not deposit medication on parts of the face remote from the nose” (see column 2, line 67-column 2 lines 1-5).

Patent '882 further claims “A method for delivering medication to a human infant or baby while allowing the infant or baby to use a soother device, the method comprising steps of:

(a) providing a nasal mask sized and configured to be placed over an infant's nose without surrounding the infant's mouth, the nasal mask being adapted to be placed in communication with a source of medication;
(b) providing a soother device adapted to be sucked on by an infant;
(c) placing the nasal mask over the infant's nose;
(d) placing the soother device in the infant's mouth; and
(e) delivering medication to the nasal mask to allow the infant to breathe in the medication through its nose while sucking on the soother device”, see claim 1.

Thus, the mask provided by '882 is merely a nose mask. Such is in contrast to the present invention in which a nose-mouth mask is provided so as to surround both the nose portion as well as the mouth portion of the user.

Another difficulty which is associated with administering medicament to infants whilst using a face mask mostly relates to poor acceptance of the mask which thus greatly decreases both the efficiency and clinical efficacy of the treatment. It is a common complaint of most parents that they find it very difficult to keep a mask tightly fitted and properly aligned to their infant's face for, more than a few seconds at a time. Noble and colleagues (Noble V, Ruggins N R, Everard M L, et al. Inhaled budesonide for chronic wheezing under 18 months of age. Arch Dis Child 1992; 67; 285-8) found that about 30% of their patients did not accept the mask while awake, and 17% did not accept it even when asleep and had to be withdrawn from a clinical study. Furthermore, it is impossible for the physician to predict which infants/children will be likely to accept the mask and which will not, thus caregivers may go to the considerable expense of acquiring an apparently suitable aerosol delivery device that is subsequently rejected by the child.

Another difficulty concerns the parents' complaints to their physician about the difficulty they are encountered while trying to apply the mask of the child or the persuasion of the child to accept the valved holding chamber with mask.

Such difficulty has discouraged physicians from using the simple, rapid to administer aerosol medication, inexpensive mask; and many of them continue to prescribe inefficient, cumbersome and very expensive small volume wet nebulizers requiring electrical power driven by a relatively noisy air compressor that requires the child to keep the mask on its face for 4-5 minutes, thus adding to caregivers' difficulty administering the medication as outlined above.

Another difficulty associated with a face mask is the fact that infants are usually provided with the medicament by a parent. In conventional devices the mother or any other health-caregiver has to hold and apply pressure to the device while aligning it with the infant's face so as to create a sealed environment. Often the health-carer holds the device and attached mask in an improper manner or applies too much pressure which leads to virtually complete failure to achieve delivery of the medicament to the child either because the child then fights the application of the mask or the mask collapses and is thus turned inside-out, making aerosol delivery impossible.

One of the major advantages of the present invention is the fact that a 80-100% hermetically sealed environment is obtained by the child with only minimal assistance by the parent or a health-carer.

The literature describes the use of alternative interfaces which do not touch the infant's face. Examples of such teaching can be found in Lotufo and colleagues (Lotufo J P, Ejzenberg B, Vieira S, et al. Continuous nebulization with terbutaline sulfate under tent inhalation: Evaluation of the efficacy in children 2 to 5 years of age in asthmatic crises. Rev Mal Respir 1998; 15: 255-61) who treated young (2-5 years old) children with asthma using a tent covering the child's head. Similarly, in an attempt to minimize environmental contamination, Wahlin and colleagues (Wahlin B, Malmstrom B, Soop M, et al. A pediatric canopy system for aerosol administration and minimised environmental pollution; Acta Anaesthesiol Scand 1996; 40 (8 pt 1): 932-9) used a hood to deliver aerosolized ribavirin to infants with respiratory syncytial virus bronchiolitis. Indeed, head canopies, or hoods, have long been used for delivery of oxygen and saline aerosols (for example, mist tents) in neonates and infants. As no face mask is required and nothing touches the face, a hood interface should provide a logical and compelling, child friendly alternative means of delivering nebulized drugs to infants. The clinical efficacy of bronchodilator or anti-inflammatory and antiallergic aerosols depends primarily on deposition in the pulmonary airways. The most direct way to study the relative efficiencies of two aerosol delivery systems is to quantify the dose and distribution of an inhaled drug using identical radiolabelled aerosols.

Another example is PCT application WO02/02052 relates to an aerosol inhalation interface. The device delivers a flow of aerosol medication to a patient breathing tidally by creating a medication rich environment proximal to the nose and mouth of the patient, which is in a reclining position. The device is suspended above the patient's nose and mouth and comprises (a) an aerosol generator for generating medicated particles; (b) a containment region, into which the generator discharges the medicated particles; (c) a downwardly projecting outlet sleeve, which is connected to the containment region and channels the particles from the containment region to create a medication rich environment proximal to the nose and mouth of the patient; and, (d) an air movement deflector, which inhibits the dispersion of the particles and disturbance of the medication rich environment.

Yet another example is EP application no. EP1402912 which relates to a pneumatic nebulizer for the delivery of medications that produces aerosol mist in a downward direction. A gas inlet introduces a high velocity gas that passes through a venturi orifice producing a venture effect. A liquid stored in a reservoir is drawn into the orifice, atomizing forming droplets. The droplets are further atomized by hitting a baffle. The mist formed substantially circumscribes the baffle.

In both said applications there is no means adapted to insure the appropriate amount of medicament is in fact inhaled by the infant. Furthermore, none of the applications above provides means for ensuring that the medicament will not reach the infant's eyes.

Recent studies (see Selley W G, Ellis R E, Flack F C, Brooks W A, Coordination of sucking, swallowing and breathing in the newborn: its relationship to infant feeding and normal development, Br J Disord Commun. 1990 December; 25(3): 311-27; Goldfield E C, Richardson M J, Lee K G, Margetts S. Coordination of sucking, swallowing, and breathing and oxygen saturation during early infant breast-feeding and bottle-feeding, Pediatr Res. 2006 October; 60(4):450-5. Epub 2006 Aug. 28; Lau C, Smith E O, Schanler R J, Coordination of suck-swallow and swallow respiration in preterm infants, Acta Paediatr. 2003 June; 92(6):721-7; Gewolb I H, Vice F L, Schwietzer-Kenney E L, Taciak V L, Bosma J F, Developmental patterns of rhythmic suck and swallow in preterm infants, Dev Med Child Neurol. 2001 January; 43(1):22-7; Gewolb I H, Vice F L, Maturational changes in the rhythms, patterning, and coordination of respiration and swallow during feeding in preterm and term infants, Dev Med Child Neurol. 2006 July; 48(7):589-94; J. S. Koenig, A. M. Davies and B. T. Thach, Coordination of breathing, sucking, and swallowing during bottle feedings in human infants, J Appl Physiol 69: 1623-1629, 1990; 8750-7587/90) have demonstrates that there is complete coordination between simultaneous breathing and sucking.

Furthermore, it was reported by Tal and colleagues (Tal A, Golan H, Grauer N, et al. Deposition pattern of radiolabeled salbutamol inhaled from a metered-dose inhaler by means of a spacer with mask in young children with airway obstruction; J Pediatr 1996; 128; 479-84) that lung deposition during crying was only about 0.35%, in contrast to an 8-fold mean of 2.5% when breathing quietly. Murakami and colleagues (Murakami G, Igarashi T, Adachi Y, et al. Measurement of bronchial hyperreactivity in infants and preschool children using a new method; Ann Allergy 1990; 64: 383-7) also reported that lung deposition in crying infants using a nebulizer and mask was negligible (scintigraphic data were provided for one patient). Wildhaber and collegues (Wildhaber J H, Dore N D, Wilson J M, et al. Inhalation therapy in asthma; nebuliser or pressurized metered-dose inhaler with holding chamber? In vivo comparison of lung deposition in children. J Pediatr 1999; 135: 28-33) recently described their experience with one crying child whose lung deposition was markedly reduced compared to his non-crying peers.

This is probably related to the fact that crying or screaming is associated with greatly prolonged expiration followed by short, high inspiratory flow velocity gasps leading to greater aerosol impaction in the throat (Janssens H M, Krijgsman A M, Brown R J, et al. Influence of tidal volume and respiratory rate on aerosol deposition in an infant upper airway model; Eur Respir J; 1999; 14(suppl 30): 179) and frequent swallowing.

By using a soothing or feeding means, the infants remain relaxed and calm. By using a soothing or feeding means while the infants are sucking the aerosol medicament can be administered in a complete coordination between the sucking of the soothing or feeding means and breathing the medicament.

Another major disadvantage to conventional masks lies in the fact that usually at least a part of the medicament is directed upwards and comes into contact with the infant's eyes. Such contact could be potentially dangerous as the eyes are particularly sensitive to some of the medicaments used. Therefore there is a long felt need for a device that would prevent the medicament from reaching and contacting the eyes of the user (especially infants). It should be stressed that this is particularly a problem with continuous wet nebulization since the aerosol medicament continues to be generated during the patient's expiration and may escape via the top of the mask. This is not likely to occur with the mask according to the present invention.

Therefore, there is a considerable need for a device interface that will increase the effective amount of the medicament that is actually inhaled by the infants and will incorporate said complete coordination while (i); ensuring an optimal seal between the device and the face of the child thus preventing the medicament from reaching the infant's eyes (ii) reducing the time the medicament remains in the spacer and thus decreasing the amount of medicament that accumulates on the walls of the aerosol reservoir; and, (iii) considerably increasing the probability that the facemask-valved holding chamber or mask-small volume wet nebulizer will be acceptable to the child, the caregiver and the child's physician by consistently adapting and sealing comfortably to the child's face, thus optimizing the therapeutic benefit.

### SUMMARY OF THE INVENTION

It is one object of the present invention to provide an infant mouth-nose mask for administering medicaments to infants, comprising:
a. a mouth portion, defining a confined mouth volume, placed over the mouth of said infant characterized by having at least one perforation reversibly coupled to calming or feeding means such that said infant remains relaxed and soothed while sucking said calming or feeding means; said confined mouth volume is characterized by volume, Vₘₒᵤₜₕ; and,
b. a nose portion, defining a confined mouth volume, placed over the nose of said infant; said nose portion is adapted to be in fluid connection with a medicament delivery source (MDS); said confined nose volume is characterized by volume, Vₙₒₛₑ;
wherein said Vₙₒₛₑ>>a*Vₘₒᵤₜₕ such that the dead-space of said mask is not greater than 28 cc.

It is another object of the present invention to provide the infant mouth-nose mask as defined above, wherein said mouth-nose mask is characterized by an external surface and an internal surface; said internal surface is adapted to come into contact with said infant's face (namely, the mouth and nose).

It is another object of the present invention to provide the infant mouth-nose mask as defined above, wherein said internal surface comprising a frame or edge surrounding the same (i.e., the internal surface of the mask).

It is another object of the present invention to provide the infant mouth-nose mask as defined above, wherein said frame is adapted to increase the cross section area which come into contact with said infant's face.

It is another object of the present invention to provide the infant mouth-nose mask as defined above, wherein said frame is adapted to enable the adjustment of said mouth-nose mask to a wide range of infant's facial cross sections.

It is another object of the present invention to provide the infant mouth-nose mask as defined above, wherein said frame is adapted to enable the adjustment of said mouth-nose mask to a wide range of infant's ages.

It is another object of the present invention to provide the infant mouth-nose mask as defined above, wherein said age is at least 1 month.

It is another object of the present invention to provide the infant mouth-nose mask as defined above, wherein said range is from at least 1 month and up to 5 years.

It is another object of the present invention to provide the infant mouth-nose mask as defined above, wherein said medicament is in the form of aerosol, gas powder or any combination thereof.

It is another object of the present invention to provide the infant mouth-nose mask as defined above, wherein said medicament is anesthetic gas selected from ethers, halogenated ethers, desflurane (2,2,2-trifluoro-1-fluoroethyl-difluoromethyl ether), sevoflurane (2,2,2-trifluoro-1-[trifluoromethyl]ethyl fluoromethyl ether), and isoflurane (2-chloro-2-(difluoromethoxy)-1,1,1-trifluoro-ethane) and any combination thereof, for use as local anesthesia, different regional anesthesia, general anesthesia.

It is another object of the present invention to provide the infant mouth-nose mask as defined above, wherein said medicament is selected from Asthma medicaments, corticosteroids, Leukotriene antagonists, beta-agonists or any combination thereof.

It is another object of the present invention to provide the infant mouth-nose mask as defined above, wherein said mask is provided for physiotherapy breathing, pulmonary rehabilitation program, acute or chronic pulmonary disorders or any combination thereof.

It is another object of the present invention to provide the infant mouth-nose mask as defined above, wherein at least a portion of said mask's external surface comprises a plurality of bulges so as to provide a rough surface.

It is another object of the present invention to provide the infant mouth-nose mask as defined above, wherein at least a portion of said mask's external surface is coarse.

It is another object of the present invention to provide the infant mouth-nose mask as defined above, additionally comprising at least one adaptor reversibly coupled to said nose portion, comprising at least one aperture for enabling exhaled medicament to exit said mouth-nose mask in a predetermined direction.

It is another object of the present invention to provide the infant mouth-nose mask as defined above, wherein said adaptor is integrated within said nose portion of said mask.

It is another object of the present invention to provide the infant mouth-nose mask as defined above, wherein said exhaled medicament exits said mouth-nose mask by means of said at least one aperture in said predetermined direction substantially without reaching said infant's eye region.

It is another object of the present invention to provide the infant mouth-nose mask as defined above, wherein nose portion is characterized by a back side, adapted to be placed upon said nose of said infant and a front side.

It is another object of the present invention to provide the infant mouth-nose mask as defined above, wherein said front said is at least partially reversibly coupled to a spacer, aerosol powder inhaler, dry powder delivery systems, nebulizer or any medicament delivery source (MDS).

It is another object of the present invention to provide the infant mouth-nose mask as defined above, wherein said front face of said nose portion comprise an internally folded rim.

It is another object of the present invention to provide the infant mouth-nose mask as defined above, wherein said internally folded rim is adapted to enable reversible coupling of at least one selected from a group consisting of a spacer, aerosol powder inhaler, dry powder delivery systems, nebulizer or any medicament delivery source (MDS) to said nose portion.

It is another object of the present invention to provide the infant mouth-nose mask as defined above, wherein said adaptor comprises a perforated disc for coupling said adaptor to a cap-like retainer to be fitted upon or around the users head.

It is another object of the present invention to provide an infant mouth-nose mask for administering medicaments to infants whilst substantially preventing said medicament from reaching the eye region of said infants, comprising:
a. a mouth portion defining a confined mouth volume, placed over the mouth of said infant characterized by having at least one perforation reversibly coupled to calming or feeding means such that said infant remains relaxed and soothed whilst sucking said calming or feeding means; said confined mouth volume is characterized by volume, Vₘₒᵤₜₕ; and,
b. a nose portion, defining a confined nose volume placed over the nose of said infant; said nose portion being adapted to be in fluid connection with a medicament delivery source (MDS); said confined nose volume is characterized by volume, Vₙₒₛₑ;
c. at least one adaptor reversibly coupled to said nose portion, comprising at least one aperture for enabling exhaled medicament to exit said mouth-nose mask in a predetermined direction;
   wherein said exhaled medicament exits said mouth-nose mask in said predetermined direction substantially without reaching said infant's eye-region.

It is another object of the present invention to provide the infant mouth-nose mask as defined above, wherein said Vₙₒₛₑ>>a*Vₘₒᵤₜₕ such that the dead-space of said mask is not greater than 28 cc, and a is in the range of about 1.5 to about 2.

It is another object of the present invention to provide the infant mouth-nose mask as defined above, wherein said adaptor is integrated within said nose portion of said mask.

It is another object of the present invention to provide the infant mouth-nose mask as defined above, wherein said mask when fitted is adapted to provide a 80-100% hermetic sealing between said mask and said mouth and nose of said infant whilst said infant sucks said calming or feeding means.

It is another object of the present invention to provide the infant mouth-nose mask as defined above, wherein at least a portion of the contours of the side-walls of said mask is accordion-like shaped so as to provide said 80-100% hermetic sealing while minimizing the mask dead-space.

It is another object of the present invention to provide the infant mouth-nose mask as defined above, wherein said medicament is in the form of aerosol, gas powder or any combination thereof.

It is another object of the present invention to provide the infant mouth-nose mask as defined above, wherein said medicament is anesthetic gas selected from ethers, halogenated ethers, desflurane (2,2,2-trifluoro-1-fluoroethyl-difluoromethyl ether), sevoflurane (2,2,2-trifluoro-1-[trifluoromethyl]ethyl fluoromethyl ether), and isoflurane (2-chloro-2-(difluoromethoxy)-1,1,1-trifluoro-ethane) and any combination thereof, for use as local anesthesia, different regional anesthesia, general anesthesia.

It is another object of the present invention to provide the infant mouth-nose mask as defined above, wherein said medicament is selected from Asthma medicaments, corticosteroids, Leukotriene antagonists, beta-agonists or any combination thereof.

It is another object of the present invention to provide the infant mouth-nose mask as defined above, wherein said mask is provided for physiotherapy breathing, pulmonary rehabilitation program, acute or chronic pulmonary disorders or any combination thereof.

It is another object of the present invention to provide the infant mouth-nose mask as defined above, wherein said calming or feeding means are selected from a group consisting of pacifier, straw, sippy attachment or a bottle's nipple already familiar to said infant.

It is another object of the present invention to provide the infant mouth-nose mask as defined above, wherein said pacifier is integrated within said mouth portion of said mask.

It is another object of the present invention to provide the infant mouth-nose mask as defined above, wherein the proportion of infants accepting said mouth-nose mask is more than about 50% according to the VT acceptance scale.

It is another object of the present invention to provide the infant mouth-nose mask as defined above, wherein the amount of time the infant remains soothed is at least 20 sec, most preferably several minutes according to VTT scale.

It is another object of the present invention to provide the infant mouth-nose mask as defined above, wherein the amount of time the infant remains soothed is increased by one stage of the BA scale.

It is another object of the present invention to provide the infant mouth-nose mask as defined above, wherein said mask additionally comprises flavor or smell enhancement material embedded within and/or coating said mask.

It is another object of the present invention to provide the infant mouth-nose mask as defined above, wherein said mask when fitted provides an internal negative pressure higher than about 5 mbar and lower than about 10 mbar, for a period of time t, whilst said infant sucks said calming or feeding means such that
(i) a 80-100% hermetic sealing between said mask and said infant mouth and nose during said period of time t is obtained;
(ii) the amount of time t needed to empty out said spacer from said medicament normalized by the physical volume of said spacer is reduced by at least 30%; so as the amount M of said medicament that accumulates on the walls of said spacer is reduced by at least 20% with respect to conventional masks; and,
(iii) the effective amount C of said medicament administered to said infant is at least 1.25 times greater than the standard currently available administering devices as measured by labeling methods.

It is another object of the present invention to provide the infant mouth-nose mask as defined above, wherein said labeling method is selected from a group selected from radio labeling methods, various marking methods, chemical labeling methods or any combination thereof.

It is another object of the present invention to provide the infant mouth-nose mask as defined above, wherein said calming or feeding means is a woman's breast nipple such that said mouth portion is adapted to be reversibly coupled to the contour of a woman's breast and said perforation is adapted to be reversibly coupled to said woman's breast nipple.

It is another object of the present invention to provide an administering apparatus for effectively administrating medicaments to infants, while substantially preventing said medicament from reaching the eyes region of said infant; said apparatus comprising:
a. a mouth-nose mask having (i) a mouth portion placed over the mouth of said infant; said mouth portion comprising at least one perforation adapted to be reversibly coupled to calming and/or feeding means such that said infant remains relaxed and soothed whilst sucking said calming or feeding means; (ii) a nose portion placed over the nose of said infant; and, (iii) at least one adaptor comprising at least one exit passage for enabling exhaled medicament to exit said mouth-nose mask in a predetermined direction;
b. at least one medicament delivery source (MDS), in fluid connection with said nose portion;
c. calming or feeding means at reversibly mounted via said mouth portion into said infant's mouth; adapted to be sucked by said infant such that said infant remains soothed and calm and thus breathing normally whilst sucking said calming or feeding means; and,
d. at least one container in fluid connection with said MDS, containing aerosol medicament to be administered to said infant;
   wherein said exhaled medicament exits said mouth-nose mask in said predetermined direction substantially without reaching said infant's eye region.

It is another object of the present invention to provide the administering apparatus as defined above, wherein said mask when fitted is adapted to provide a 80-100% hermetic seal between said mask and said infant's mouth and nose whilst said infant sucks said nipple.

It is another object of the present invention to provide the administering apparatus as defined above, wherein said MDS is selected from spacer, valved aerosol holding chamber, wet nebulizer, aerosol powder inhaler, anesthetic drugs as liquid, aerosols, or gas, dry powder delivery systems or any combination thereof.

It is another object of the present invention to provide the administering apparatus as defined above, wherein the proportion of infants accepting said mouth-nose mask is more than about 50% according to the VT acceptance scale.

It is another object of the present invention to provide the administering apparatus as defined above, wherein the amount of time the infant remains soothed is at least 20 sec, most preferably several minutes according to VTT scale.

It is another object of the present invention to provide the administering apparatus as defined above, wherein the amount of time the infant remains soothed is increased by one stage of the BA scale.

It is another object of the present invention to provide the administering apparatus as defined above, wherein said wherein said mask additionally comprises flavor or smell enhancement material embedded within and/or coating said mask.

It is another object of the present invention to provide the administering apparatus as defined above, wherein said mask, when fitted provides an internal negative pressure higher than about 5 mbar and lower than about 10 mbar, for a period of time t, whilst said infant sucks said nipple such that:
a. a 80-100% hermetic sealing between said mask and said infant mouth and nose during said period of time t is obtained;
b. the amount of time t needed to empty out said spacer from said medicament normalized by the physical volume of said spacer is reduced by at least 25%; so that the amount M of said medicament that accumulates on the walls of said spacer is reduced by at least 25% with respect to conventional delivery systems; and,
c. the effective amount C of said medicament administered to said infant is at least 1.25, times greater than the standard administering devices as measured by labeling methods.

It is another object of the present invention to provide the administering apparatus as defined above, wherein said labeling method is selected from a group selected from radio labeling methods, different marking methods, chemical labeling methods or any combination thereof.

It is another object of the present invention to provide the administering apparatus as defined above, wherein said calming or feeding means is a woman's breast nipple such that said mouth portion is adapted to be reversibly coupled to the contour of a woman's breast and said perforation is adapted to be reversibly coupled to said woman's nipple.

It is another object of the present invention to provide the administering apparatus as defined above, wherein at least a portion of said mask's external surface comprises a plurality of bulges so as to provide a rough surface.

It is another object of the present invention to provide the administering apparatus as defined above, wherein at least a portion of said mask's external surface is coarse.

It is another object of the present invention to provide the administering apparatus as defined above, wherein said mouth-nose mask is characterized by an external surface and an internal surface; said internal surface is adapted to come into contact with said infant's face.

It is another object of the present invention to provide the administering apparatus as defined above, wherein said internal surface comprising a frame or edge surrounding the same.

It is another object of the present invention to provide the administering apparatus as defined above, wherein said frame is adapted to increase the cross section area which come into contact with said infant's face.

It is another object of the present invention to provide the administering apparatus as defined above, wherein said frame is adapted to enable the adjustment of said mouth-nose mask to a wide range of infant's facial cross sections.

It is another object of the present invention to provide the administering apparatus as defined above, wherein said frame is adapted to enable the adjustment of said mouth-nose mask to a wide range of infant's ages.

It is another object of the present invention to provide the administering apparatus as defined above, wherein said age is at least 1 month.

It is another object of the present invention to provide the administering apparatus as defined above, wherein said range is from at least 1 month to at least 5 years.

It is another object of the present invention to provide the administering apparatus as defined above, wherein nose portion is characterized by a back side, adapted to be placed upon said nose of said infant and a front side.

It is another object of the present invention to provide the administering apparatus as defined above, wherein said front said is at least partially reversibly coupled to a spacer, aerosol powder inhaler, dry powder delivery systems, nebulizer or any medicament delivery source (MDS).

It is another object of the present invention to provide the administering apparatus as defined above, wherein said front face of said nose portion comprise an internally folded rim.

It is another object of the present invention to provide the administering apparatus as defined above, wherein said internally folded rim is adapted to enable reversible coupling of at least one selected from a group consisting of a spacer, aerosol powder inhaler, dry powder delivery systems, nebulizer or any medicament delivery source (MDS) to said nose portion.

It is another object of the present invention to provide the administering apparatus as defined above, wherein said adaptor comprises a perforated disc for coupling said adaptor to a cap-like retainer to be fitted upon or around the users head.

It is another object of the present invention to provide the administering apparatus as defined above, wherein said medicament is in the form of aerosol, gas powder or any combination thereof.

It is another object of the present invention to provide the administering apparatus as defined above, wherein said medicament is anesthetic gas selected from ethers. halogenated ethers, desflurane (2,2,2-trifluoro-1-fluoroethyl-difluoromethyl ether), sevoflurane (2,2,2-trifluoro-1-[trifluoromethyl]ethyl fluoromethyl ether), and isoflurane (2-chloro-2-(difluoromethoxy)-1,1,1-trifluoro-ethane) and any combination thereof, for use as local anesthesia, different regional anesthesia, general anesthesia.

It is another object of the present invention to provide the administering apparatus as defined above, wherein said medicament is selected from Asthma medicaments, corticosteroids, Leukotriene antagonists, beta-agonists or any combination thereof.

It is another object of the present invention to provide the administering apparatus as defined above, wherein said mask is provided for physiotherapy breathing, pulmonary rehabilitation program, acute or chronic pulmonary disorders or any combination thereof.

It is another object of the present invention to provide a method for administering medicaments to infants while preventing said medicament from reaching the eye region of said infant. The method comprises steps selected inter alia from:
a. providing an administering apparatus, comprising:
   i. a mouth-nose mask having (i) a mouth portion, defining a confined mouth volume, placed over the mouth of said infant; said mouth portion comprising at least one perforation reversibly coupled to calming or feeding means such that said infant remains soothed whilst sucking said calming or feeding means; (ii) a nose portion, defining a confined nose volume, placed over the nose of said infant; and, (iii) at least one adaptor comprising at least aperture for enabling exhaled medicament to exit said mouth-nose mask in a predetermined direction;
   ii. at least one medicament delivery source (MDS), in fluid connection with said nose portion;
   iii. calming or feeding means at least reversibly mounted via said mouth portion into said infant's mouth; adapted to be sucked by said infant such that said infant remains soothed while sucking said calming or feeding means; and,
   iv. at least one container in fluid connection with said MDS, containing a medicament to be administered to said infant;
b. reversibly coupling said calming or feeding means to said perforation within said mouth portion;
c. gently placing said mouth portion over the infant's mouth so as to surround said mouth of said infant and define a confined mouth volume;
d. gently placing said nose portion over the infant noise so as to surround said nose of said infant and define a confined n volume;
e. reversibly coupling said MDS to said nose portion;
f. reversibly coupling said at least one container to said MDS;
g. efficiently administering said medicament to said infant by enabling said infant to suck said calming or feeding means whilst fully coordinating breathing said medicament; and,
h. directing the exhaled medicament from said mouth-nose mask in said predetermined direction by means of said at least one aperture, thereby substantially preventing said exhaled medicament from reaching said eye region of said infant.

It is another object of the present invention to provide the method as defined above, wherein said step d is preformed prior to said step c.

It is another object of the present invention to provide the method as defined above, wherein said steps of d and c are performed simultaneously.

It is another object of the present invention to provide the method as defined above, wherein said adaptor is integrated within said nose portion of said mask.

It is another object of the present invention to provide the method as defined above, wherein said step (f) of efficiently administering said medicament is performed such that a 80-100% hermetic sealing between said mask and said infant mouth and nose is achieved.

It is another object of the present invention to provide the method as defined above, additionally comprising the step of selecting said calming or feeding means from a group consisting of pacifier, straw, sippy mouthpiece or a bottle already familiar to said infant.

It is another object of the present invention to provide the infant mouth-nose mask as defined above, wherein said pacifier is integrated within said mouth portion of said mask.

It is another object of the present invention to provide the method as defined above, additionally comprising the step of coating and/or embedding flavor or smell enhancement material within said mask.

It is another object of the present invention to provide the method as defined above, additionally comprising a step of selecting said calming or feeding means from a group consisting of a woman's breast nipple; such that said mouth portion is reversibly coupled to the contour of a woman's breast and said perforation is reversibly coupled to said woman's nipple.

It is another object of the present invention to provide the method as defined above, wherein said confined nose volume is characterized by volume, Vₙₒₛₑ; said confined mouth volume is characterized by volume, Vₘₒᵤₜₕ; such that said Vₙₒₛₑ>>a*Vₘₒᵤₜₕ such that the dead-space of said mask is not greater than 28 cc, and a is in the range of about 1.5 to about 2.

It is another object of the present invention to provide the method as defined above, additionally comprising step of provides at least a portion of said mask's external surface with a plurality of bulges so as to provide a rough surface.

It is another object of the present invention to provide the method as defined above, additionally comprising step of providing at least a portion of said mask's external surface coarse.

It is another object of the present invention to provide the method as defined above, wherein said mouth-nose mask is characterized by an external surface and an internal surface; said internal surface is adapted to come into contact with said infant's face.

It is another object of the present invention to provide the method as defined above, wherein said internal surface comprising a frame or edge surrounding the same.

It is another object of the present invention to provide the method as defined above, wherein said frame is adapted to increase the cross section area which come into contact with said infant's face.

It is another object of the present invention to provide the method as defined above, wherein said frame is adapted to enable the adjustment of said mouth-nose mask to a wide range of infant's facial cross sections.

It is another object of the present invention to provide the method as defined above, wherein said frame is adapted to enable the adjustment of said mouth-nose mask to a wide range of infant's ages.

It is another object of the present invention to provide the method as defined above, wherein said age is at least 1 month.

It is another object of the present invention to provide the method as defined above, wherein said range is from at least 1 month to at least 5 years.

It is another object of the present invention to provide the method as defined above, wherein nose portion is characterized by a back side, adapted to be placed upon said nose of said infant and a front side.

It is another object of the present invention to provide the method as defined above, wherein said front said is at least partially reversibly coupled to a spacer, aerosol powder inhaler, dry powder delivery systems, nebulizer or any medicament delivery source (MDS).

It is another object of the present invention to provide the method as defined above, wherein said front face of said nose portion comprise an internally folded rim.

It is another object of the present invention to provide the method as defined above, wherein said internally folded rim is adapted to enable reversible coupling of at least one selected from a group consisting of a spacer, aerosol powder inhaler, dry powder delivery systems, nebulizer or any medicament delivery source (MDS) to said nose portion.

It is another object of the present invention to provide the method as defined above, wherein said front face of said nose portion comprise an internally folded rim.

It is another object of the present invention to provide the method as defined above, wherein said internally folded rim is adapted to enable reversible coupling of at least one selected from a group consisting of a spacer, aerosol powder inhaler, dry powder delivery systems, nebulizer or any medicament delivery source (MDS) to said nose portion.

It is another object of the present invention to provide the method as defined above, wherein said adaptor comprises a perforated disc for coupling said adaptor to a cap-like retainer to be fitted upon or around the users head.

It is another object of the present invention to provide the method as defined above, wherein said medicament is in the form of aerosol, gas powder or any combination thereof.

It is another object of the present invention to provide the administering apparatus as defined above, wherein said medicament is anesthetic gas selected from ethers [symbol] halogenated ethers, desflurane (2,2,2-trifluoro-1-fluoroethyl-difluoromethyl ether), sevoflurane (2,2,2-trifluoro-1-[trifluoromethyl]ethyl fluoromethyl ether), and isoflurane (2-chloro-2-(difluoromethoxy)-1,1,1-trifluoro-ethane) and any combination thereof, for use as local anesthesia, different regional anesthesia, general anesthesia.

It is another object of the present invention to provide the method as defined above, wherein said medicament is selected from Asthma medicaments, corticosteroids, Leukotriene antagonists, beta-agonists or any combination thereof.

It is another object of the present invention to provide the method as defined above, wherein said mask is provided for physiotherapy breathing, pulmonary rehabilitation program, acute or chronic pulmonary disorders or any combination thereof.

It is still an object of the present invention to provide a method for minimizing the rejection by an infant of the mask interface between the aerosol source and the infant's airway. The method comprises steps selected inter alia from:
a. providing an administering apparatus comprising:
   i. a mouth-nose mask having (i) a mouth portion placed over the mouth of said infant; said mouth portion comprising at least one perforation reversibly coupled to calming or feeding means such that said infant remains soothed whilst sucking said calming or feeding means; (ii) a nose portion placed over the nose of said infant; and, (iii) at least one adaptor comprising at least one exit passage for enabling exhaled medicament to exit said mouth-nose mask in a predetermined direction;
   ii. at least one medicament generation and delivery source (MDS), in fluid connection with said nose portion;
   iii. calming or feeding means at least reversibly mounted via said mouth portion into said infant's mouth; adapted to be sucked by said infant such that said infant remains soothed whilst sucking said calming or feeding means; and,
   iv. at least one container in fluid connection with said MDS, containing a medicament to be administered to said infant;
b. at least partially reversibly coupling said calming or feeding means to said perforation within said mouth portion;
c. gently placing said mouth portion over the infant's mouth; and said nose portion over the infant's noise;
d. at least partially reversibly coupling said MDS to said nose portion;
e. reversibly coupling said at least one container and/or feeding source to said MDS;
f. increasing the acceptance of said infant to receive said medicament, by selecting said calming or feeding means from a group consisting of pacifier, a bottle, a sippy cup mouthpiece or a straw already known to said infant or a woman's nipple; and,
g. efficiently administering said medicament to said infant;
   wherein the proportion of infants accepting said mouth-nose mask is more than about 50% according to the VT acceptance scale.

It is still an object of the present invention to accomplish a minimal mask dead space in a gentle and comfortable manner for both the infant and caregiver by providing a sealed environment between the child's face and the mask by means of atmospheric pressure resulting from the creation of subatmospheric pressure within the mask produced by the sucking action of the child.

It is lastly an object of the present invention to provide the method as defined above, additionally comprising step of coating and/or embedding flavor or smell enhancement material within said mask.

It is still an object of the present invention to provide a universal adaptor adapted to be coupled to a nose-mouth mask and deliver medicament to an infant, comprising (a) at least one nose end, adapted to be reversibly coupled to said nose portion of a nose-mouth mask; and, (b) at least one MDS end, adapted to be reversibly coupled to at least one MDS; wherein said at least one nose end comprising at least one aperture for enabling an exhaled medicament to exit said nose portion of said nose-mouth mask in a predetermined direction. It is still an object of the present invention to provide the universal adaptor as defined above, wherein said exhaled medicament exits said mouth-nose mask in said predetermined direction substantially without reaching said infant's eye-region.

### BRIEF DESCRIPTION OF THE FIGURES

In order to understand the invention and to see how it may be implemented in practice, a plurality of embodiments will now be described, by way of non-limiting examples only, with reference to the accompanying drawings, in which

FIG. 1 illustrates the infant mouth-nose mask 10 according to a preferred embodiment of the present invention.

FIGS. 2A-2B illustrate the mask 10 prior to and after the suction of the calming or feeding means by the infant.

FIG. 3 illustrates the infant mouth-nose mask 10 coupled to a woman's breast nipple.

FIGS. 4-6 illustrate the administering apparatus 100 according to a preferred embodiment of the present invention.

FIGS. 6A and 6B illustrate another preferred embodiment of the present invention according to which the mask is coupled to feeding means 30 (e.g., straw).

FIG. 7 illustrates the medicament flow within the administering apparatus 100.

FIG. 8 illustrates the dedicated adaptor 80 coupled to nose portion of the mouth-nose mask 10.

FIGS. 9 and 10 illustrate a cross sectional view of mask 10 coupled to the adaptor 80.

FIGS. 11-12 illustrate different views of the mask 10.

FIG. 13 illustrates another embodiment of the nose-mouth mask 10.

FIGS. 14-20 illustrate different masks that were used in the dead space volume testing.

FIGS. 21-24 illustrate the results of the acceptance evaluation of the mask as provided by the present invention by children.

### DETAILED DESCRIPTION OF THE PREFERRED EMBODIMENTS

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide an active mask that facilitates drugs and medicament administering to infants.

It is one object of the present invention to provide an infant mouth-nose mask for administering medicaments to infants, comprising:
c. a mouth portion, defining a confined mouth volume, placed over the mouth of said infant characterized by having at least one perforation reversibly coupled to calming or feeding means such that said infant remains relaxed and soothed while sucking said calming or feeding means; said confined mouth volume is characterized by volume, Vₘₒᵤₜₕ; and,
d. a nose portion, defining a confined mouth volume, placed over the nose of said infant; said nose portion is adapted to be in fluid connection with a medicament delivery source (MDS); said confined nose volume is characterized by volume, Vₙₒₛₑ;
   wherein said Vₙₒₛₑ>>a*Vₘₒᵤₜₕ such that the dead-space of said mask is not greater than 28 cc.

It is another object of the present invention to provide the infant mouth-nose mask as defined above, additionally comprising at least one adaptor reversibly coupled to said nose portion, comprising at least one aperture for enabling exhaled medicament to exit said mouth-nose mask in a predetermined direction.

It is another object of the present invention to provide the infant mouth-nose mask as defined above, wherein said adaptor is integrated within said nose portion of said mask.

It is another object of the present invention to provide the infant mouth-nose mask as defined above, wherein said exhaled medicament exits said mouth-nose mask by means of said at least one aperture in said predetermined direction substantially without reaching said infant's eye region.

The term “about” refers hereinafter to a range of 25% below or above the referred value.

The term “infants” or “toddlers” refers hereinafter to any a human child at the youngest stages of life.

The term “medicament effective amount” refers hereinafter to the actual amount of medicament inhaled by the infant.

The term “physiotherapy breathing” refers hereinafter to any chest physiotherapy breathing treatments/exercises. Such treatments are recommended for patients suffering from acute and chronic breathing problems such as lung disease, central nervous system disorders such as acute, chronic or progressive myopathic or neuropathy, and other problems that lead to impaired lung functioning.

These exercises involve certain techniques to decrease shortness of breath; facilitate breathing, coughing and mucus removal; and train the lungs to perform more effectively.

Thus, said breathing exercises are commonly incorporated into the overall pulmonary rehabilitation program of patients with acute or chronic pulmonary disorders. Breathing exercises are designed to restrain the muscles of respiration and improve or redistribute ventilation, lessen the work of breathing, and improve the gas exchange and oxygenation. Active range of motion exercises, to the shoulders and trunk also help expand the chest, facilitate deep breathing, and often stimulate the cough reflex.

The term “Video Tracing (VT) acceptance scale” refers hereinafter to an acceptance scale constructed by observation and video documentation of administering medicament to infants whilst using a standard device and whilst using the device and mask according to the present invention. The VT acceptance scale is based on statistical analysis of the video documentation; and will provide a quantitative index for the number of infant's accepting the mask/device.

The VT acceptance scale will indicate the number of infants that accepted the medicament by allowing the mask, as provided by the present invention, to be fitted to their face with little or no resistance on the part of the child.

The “VT acceptance scale” will incorporate within the statistical analysis parameters selected from a group consisting of the amount of time the device has been retained over the infant's face, the amount of time the infant cried, the voice, sound and volume of the infant's crying.

The term “Video Tracing Time (VTT) acceptance scale” refers hereinafter to a time scale that will indicate the amount of time the infant remains calm and soothed whilst the mask, as provided by the present invention, is fitted to the face. The VTT scale will be constructed by observation and video documentation of administering medicament to infants whilst using a standard device and while using the device and mask according to the present invention. The VTT scale will be based on statistical analysis of the video documentation and will provide a quantitative index for the amount of time the infant remains calm and soothed while fitted with the mask and aerosol generation/delivery device.

The term “Behavioural assessment scale (BAS)” refers herein after to the scale provided by I Amirav, I Balanov, M Gorenberg, D Groshar, A S Luder in Arch Dis Child 2003; 88: 719-723. The BAS scale was obtained as follows: during treatment, infants were observed every minute. One point was scored for every minute that the infant either cried or resisted the treatment for more than 20 seconds, more preferably several minutes. Finally, a behavioral index of 6 within the scale represented maximal distress whereas index 0 represented no distress.

The term “full coordination” refers hereinafter to the act of coordinating i.e., enabling two different operations to completely work together to obtain a goal or effect. In the present invention a full coordination between sucking the calming means by the infant and breathing is obtained. By this full coordination, the infant is better able to more efficiently inhale the medicament so that the effective amount of the medicament inhaled by the infant is increased and the mask is fully accepted by the infant without crying or apparently wishing to remove the mask.

The term “about” refers hereinafter to a range of 25% below or above the reference value.

The term “Radioisotopic labeling” or “radiolabeling” refers hereinafter to a technique for tracking the passage of a sample of substance through a system. The substance is “labelled” by including radionuclides in its chemical composition. Their presence can be determined by detecting the radiation they emit by means of a scintillation camera.

The present invention provides a device and method for effectively administering medicament to infants whilst (i) preventing the medicament given to the infant from reaching the infant's eye region; and, (ii) gently and acceptably providing a complete hermetic seal between the mask and the infant's mouth area and nose area.

The mask is provided with a frame or edge surrounding the back/-internal side of the mask (or in other words, a rim). Such rim is utilized to better seal the mask and to enable the adjustment of the mask to a wide range of facial cross sections. In other words, the mask will fit to an infant of several months and will accompany him till he will be of several years of age, especially from the age of a month to the age of 28 months.

Furthermore, the mask is configured such that the ‘Dead-Space’ is minimized, yet still medication can reach the infant's mouth. Thus, even when an infant has a cold—he will be able to breath through the mouth.

Such a configuration is enabled due to the special design of the mask in which the volume of the mouth portion is minimized.

One of the major limitations of administering aerosol medicament to infants is that the effective amount (i.e. the actual amount) of medicament inhaled by the infant and delivered to the infant's lungs is not substantial and practically negligible even under the best of circumstances.

The following table, table 1, represents an overview of various studies carried out with several devices in young children and their medicament effective amount (as measured by radio labeling methods):

TABLE 1
medicament effective amount

| | | | | | | Lung | |
|---|---|---|---|---|---|---|---|
| | | | | | | deposition or | |
| | | | | | Aerosol | medicament | |
| | | Age | | No. | Delivery | effective | Deposition |
| No. | Reference | (range) | Disease | Subjects | device | amount | expressed as |
| 1 | Wildhaber | <4 yr | Stable | 8 | pMDI-BH | 5.4% | % of total |
| | (see | | asthma | | (DC) | | actuated |
| | reference 1) | | | | | | dose |
| 2 | Tal (see | 3 mo-5 yr | 7 asthma, | 15 | pMDI-AC | 2% | % of inhaled |
| | reference 2) | | 4 CF, 4 | | (non- | | drug |
| | | | BPD | | DC) | | |
| 3 | Amirav (see | 1-14 mo | Acute | 12 | Nebulizer | 1.5% in | % of |
| | reference 3) | | RSV | | | right lung | nebulizer |
| | | | | | | | output |
| 4 | Mallol (see | 3-24 mo | CF | 20 | Nebulizer | 2% | % of total |
| | reference 4) | | | | using 2 | | dose placed |
| | | | | | different | | in nebulizer |
| | | | | | particle | | |
| | | | | | sizes | | |
| 5 | Chua (see | 4-15 mo | CF | 12 | Nebulizer | 1.3% | % of |
| | reference 5) | | | | | | nebulizer |
| | | | | | | | output |

AC: Aerochamber,
BH: Babyhaler,
DC: detergent-coated,
CF: cystic fibrosis,
BDP: bronchopulmonary dysplasia,
RSV: respiratory syncytial virus.

### REFERENCES

1. Wildhaber J H, Janssens H M, Pierart F, Dore N D, Devadason S G, LeSouef P N. High-percentage lung delivery in children from detergent-treated spacers. Pediatr Pulmonol 2000; 29(5):389-93.
2. Tal A, Golan H, Grauer N, Aviram M, Albin D, Quastel M R. Deposition pattern of radiolabeled salbutamol inhaled from a metereddose inhaler by means of a spacer with mask in young children with airway obstruction. J Pediatr 1996; 128(4):479-84.
3. Amirav I, Balanov I, Gorenberg M, Luder A S, Newhouse M T, Groshar D. Beta-agonist aerosol distribution in respiratory syncytial virus bronchiolitis in infants. J Nucl Med 2002; 43(4):487-91.
4. Mallol J, Rattray S, Walker G, Cook D, Robertson C F. Aerosol deposition in infants with cystic fibrosis. Pediatr Pulmonol 1996; 21(5):276-81.
5. Chua H L, Collis G G, Newbury A M, Chan K, Bower G D, Sly P D, et al. The influence of age on aerosol deposition in children with cystic fibrosis. Eur Respir J 1994; 7(12):2185-91.

As can be seen from table 1, the effective amount of medicament inhaled by the infant is practically negligible. Therefore, one object of the present invention is to provide a device and method that is especially adapted to optimize the effective amount of the medicament reliably and predictably inhaled by the infant and deposited in the lungs. Hence, one main object of the present invention is to provide a device and method that will substantially increase the average medicament effective amount.

Another difficulty associated with administering aerosol medicament to infants is the fact that infants are usually provided with the medicament with the aid of the mother or other care giver. With conventional devices the mother or any other health-carer must firmly hold the mask and place against the infant's face so as to create a sealed environment, (in effect achieving this by very variable and not standardized force). Furthermore, the health-carer often holds the device in an improper manner which leads to a poor mask to face seal and leaks of medicament from the device.

Therefore, another object of the present invention is to provide a 80-100% hermetically sealed environment without the need to use pressure or force to achieve a seal between the mask and the infant's face.

Another major feature of the mask as provided by the present invention is the fact that the mask reduces, considerably the likelihood that the medicaments will significantly contact the eyes. Since the eye region is a highly sensitive region, it is desired that said region will be protected from contamination of medications.

Another major advantage of the present invention is the fact that the acceptance of infants to inhale the medicament or even to allow the parents or physician to apply the device (i.e., the mask) on them is enhanced. This is achieved simply by applying first the mask containing merely the relaxing/calming means (i.e., the pacifier or a bottle's nipple already known to the infant), thus the infant is becoming familiar with the mask and hence will not reject when the mask is combined with a medicament delivery source (MDS) (e.g., spacer, nebulizer).

It is another object of the present invention to provide the infant mouth-nose mask as defined above, wherein said pacifier is integrated within said mouth portion of said mask.

It is another object of the present invention to provide an infant mouth-nose mask for administering medicaments to infants while substantially preventing said medicament from reaching the eye region of said infants, comprising:
a. a mouth portion, defining a confined mouth volume, placed over the mouth of said infant characterized by having at least one perforation reversibly coupled to calming or feeding means such that said infant remains relaxed and soothed while sucking said calming or feeding means; said confined mouth volume is characterized by volume, Vₘₒᵤₜₕ; and,
b. a nose portion, defining a confined mouth volume, placed over the nose of said infant; said nose portion is adapted to be in fluid connection with a medicament delivery source (MDS); said confined nose volume is characterized by volume, Vₙₒₛₑ;
wherein said Vₙₒₛₑ>>a*Vₘₒᵤₜₕ such that the dead-space of said mask is not greater
than 28 cc, and a is in the range of about 1.5 to about 2.

It is another object of the present invention to provide the infant mouth-nose mask as defined above, wherein said mouth-nose mask is characterized by an external surface and an internal surface; said internal surface is adapted to come into contact with said infant's face.

It is another object of the present invention to provide the infant mouth-nose mask as defined above, wherein said internal surface comprising a frame or edge surrounding the same.

It is another object of the present invention to provide the infant mouth-nose mask as defined above, wherein said frame is adapted to increase the cross section area which come into contact with said infant's face.

It is another object of the present invention to provide the infant mouth-nose mask as defined above, wherein said frame is adapted to enable the adjustment of said mouth-nose mask to a wide range of infant's facial cross sections.

It is another object of the present invention to provide the infant mouth-nose mask as defined above, wherein said frame is adapted to enable the adjustment of said mouth-nose mask to a wide range of infant's ages.

It is another object of the present invention to provide the infant mouth-nose mask as defined above, wherein said age is at least 1 month.

It is another object of the present invention to provide the infant mouth-nose mask as defined above, wherein said range is from at least 1 month to at least 5 years.

It is another object of the present invention to provide the infant mouth-nose mask as defined above, wherein at least a portion of said mask's external surface comprises a plurality of bulges so as to provide a rough surface.

It is another object of the present invention to provide the infant mouth-nose mask as defined above, wherein at least a portion of said mask's external surface is coarsed.

It is another object of the present invention to provide the infant mouth-nose mask as defined above, additionally comprising at least one adaptor reversibly coupled to said nose portion, comprising at least one aperture for enabling exhaled medicament to exit said mouth-nose mask in a predetermined direction.

It is another object of the present invention to provide the infant mouth-nose mask as defined above, wherein said exhaled medicament exits said mouth-nose mask by means of said at least one aperture in said predetermined direction substantially without reaching said infant's eye region.

It is another object of the present invention to provide the infant mouth-nose mask as defined above, wherein nose portion is characterized by a back side, adapted to be placed upon said nose of said infant and a front side.

It is another object of the present invention to provide the infant mouth-nose mask as defined above, wherein said front said is at least partially reversibly coupled to a spacer, aerosol powder inhaler, dry powder delivery systems, nebulizer or any medicament delivery source (MDS).

It is another object of the present invention to provide the infant mouth-nose mask as defined above, wherein said front face of said nose portion comprise an internally folded rim.

It is another object of the present invention to provide the infant mouth-nose mask as defined above, wherein said internally folded rim is adapted to enable reversible coupling of at least one selected from a group consisting of a spacer, aerosol powder inhaler, dry powder delivery systems, nebulizer or any medicament delivery source (MDS) to said nose portion.

It is another object of the present invention to provide the infant mouth-nose mask as defined above, wherein said adaptor comprises a perforated disc for coupling said adaptor to a cap-like retainer to be fitted upon or around the users head.

According to another embodiment the mask as provide by the present invention is adapted to provide medicament given to the infants. According to one embodiment said medicament is anesthetic gas (e.g., ethers. halogenated ethers, desflurane (2,2,2-trifluoro-1-fluoroethyl-difluoromethyl ether), sevoflurane (2,2,2-trifluoro-1-[trifluoromethyl]ethyl fluoromethyl ether), and isoflurane (2-chloro-2-(difluoromethoxy)-1,1,1-trifluoro-ethane)) for use as local anesthesia, different regional anesthesia, general anesthesia.

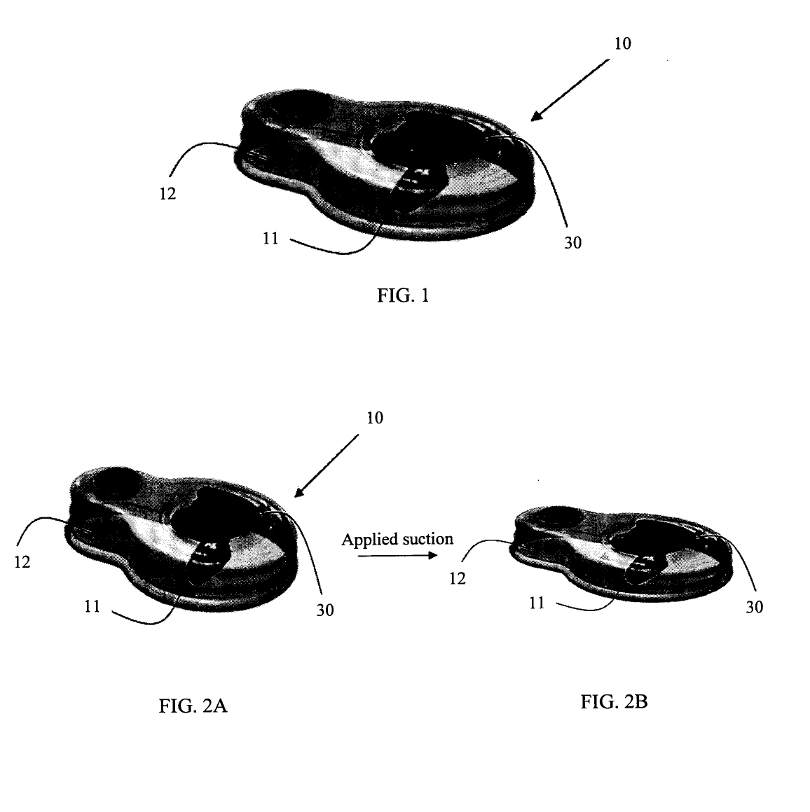

Reference is now made to FIG. 1, presenting in a non-limiting manner the infant mouth-nose mask 10 according to a preferred embodiment of the present invention. The mask and exhalation path is especially constructed and designed so as to (i) virtually prevent any medicament from reaching the infant's eye-region; (ii) provide a 80-100% hermetic seal between the mask and the infant's mouth area and nose area without the need for the caregiver to apply variable and unpredictable force; and, (iii) minimize the mask dead space, and (iv) achieve the seal between the mask and infant's face in a manner designed to calm the infant and increase the acceptability of a mask for aerosol therapy of infants.

The mask comprises at least two portions: (a) mouth portion 11, defining a confined mouth volume; and (b) a nose portion 12, defining a confined nose volume. The mouth portion is characterized by having at least one perforation 1 adapted to be reversibly coupled to relaxing/calming or feeding means 30. The calming or feeding means are selected from a group consisting of pacifier or a bottle's nipple already known to the infant. Furthermore, the relaxing or feeding means are used to soothe the infant.

It is another object of the present invention to provide the infant mouth-nose mask as defined above, wherein said pacifier is integrated within said mouth portion of said mask.

The nose portion is adapted to be placed over the infant nose and to be reversibly coupled with a dedicated adaptor reversibly coupled with a fluid connection to a medicament delivery source (MDS).

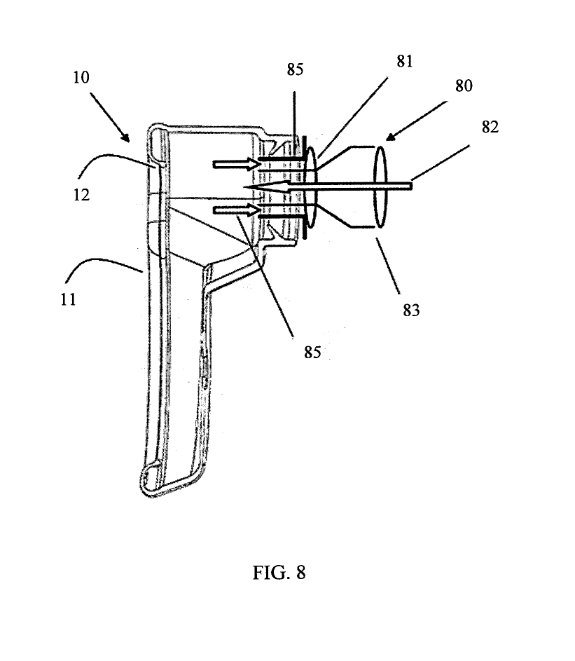

The adaptor 80 (see FIG. 8) comprising at least one exit passage for enabling exhaled medicament to exit the mouth-nose mask in a predetermined direction. Said predetermined direction prevents the medicament from reaching the infant's eye region.

It should be pointed out that the infant is provided with the medicament by complete coordination between sucking the calming or feeding means and inhaling the medicament through the nose portion of the mask.

The important advantage of the mask is the fact that it is especially configured, constructed and designed so as to facilitate the above and to enable a 80-100% hermetic sealing between the mask and the infant's mouth area and nose area when fitted and while the infant sucks said calming or feeding means.

According to one embodiment of the present invention, the 80-100% hermetic sealing will be enabled due to the accordion configuration in the contours of the mask walls. 10. Reference is now made to FIGS. 2a and 2b illustrating mask 10 prior to and after the suction of the calming or feeding means by the infant. FIG. 2a illustrates mask 10 prior to the suction and FIG. 2b illustrates the mask after suction was applied. As can be seen from the figures, due to the accordion design and configuration of the mask 10, when the infant sucks the calming or feeding means, the volume of the mask is substantially reduced so that 80-100% hermetic sealing is readily and consistently obtained by application of atmospheric pressure alone without the need for application of additional external force by the caregiver.

According to another embodiment of the present invention, the mask when fitted provides an internal negative pressure higher than about 5 mbar and lower than about 10 mbar, for a period of time t is obtained whilst the infant sucks said calming or feeding means.

Furthermore, the amount of time t needed to empty out said aerosol reservoir (spacer) from said medicament normalized by the physical volume of said spacer is reduced by at least 25% as measured by different labeling methods. The amount M of said medicament that accumulates on the walls of said spacer is reduced by at least 25% with respect to conventional masks as measured by different labeling methods.

The different labeling methods can be selected from a group consisting of radio labeling methods, different marking methods, chemical labeling methods or any combination thereof.

Moreover, the effective amount C of said medicament administered to said infant is at least 1.25 times greater than the standard administering devices as measured by labeling methods.

According to another embodiment of the present invention, the labeling methods are selected from a group selected from radio labeling methods, different marking methods, chemical labeling methods or any combination thereof.

According to one embodiment of the present invention, the calming or feeding means are selected from a group consisting of pacifier or a bottle with rubber nipple or sippy cup mouthpiece already familiar to the infant (i.e. its own pacifier/soother).

It is another object of the present invention to provide the infant mouth-nose mask as defined above, wherein said pacifier is integrated within said mouth portion of said mask.

According to yet another embodiment of the present invention, the mask is coated and/or embedded with flavor/smell enhancement material so that the infant sees the mask as something pleasant

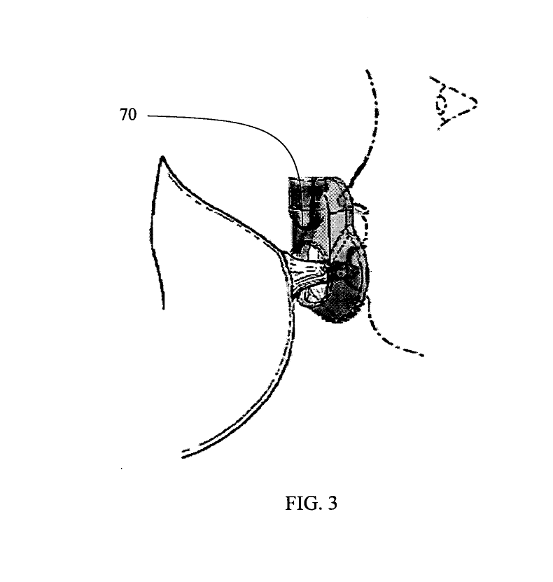

Reference is now made to FIG. 3, illustrating the mouth-nose mask coupled to a woman's breast nipple 70.

One of the major advantages of the present invention is the ability to couple the mouth portion within the mouth-nose mask 10 to a woman's nipple. i.e., using breast feeding as the calming and feeding means. The great advantage of using a woman's breast nipple is the fact that the breast-feeding process, in general, and a woman's nipple, in particular, is one of the most relaxing activities for infants. Therefore, combining the breast-feeding process or the woman's breast nipple within a medicament administering device (i.e., coupling the mask to the nipple) may be highly advantageous. According to said embodiment, the mouth portion is adapted to be reversibly coupled to the contour of a woman's breast and said perforation is adapted to be reversibly coupled to the woman's nipple.

According to this embodiment, the nose portion is reversibly coupled to the adaptor 80 (as will be further elaborated in the description of FIG. 8).

According to another embodiment of the present invention, the amount or proportion of infants accepting the mouth-nose mask is more than about 50% according to the VT acceptance scale.

According to another embodiment of the present invention, the amount of time the infant remains relaxed and soothed is at least 20 sec, more preferably several minutes (approx. 5-10 minutes) according to VTT scale.

According to another embodiment of the present invention, the amount of time the infant remains calmed and soothed is increased by one stage of the BA scale.

It should be pointed out that the mask will be made from silicone or any other highly flexible and safe material.

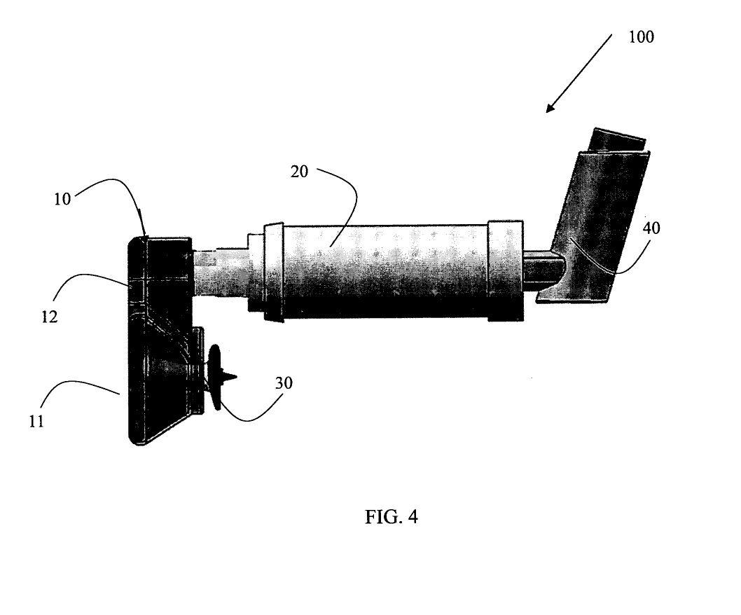

Reference is now made to FIG. 4, presenting in a non-limiting manner the administering apparatus 100 adapted to efficiently administer medicament to infants.

As described above, the three major difficulties associated with administering medicament to infants are:

(a) practically negligible effective amount of medicament inhaled by infants;
(b) no adequate, seal is consistently obtained between the mask and the infant's face;
(c) a relatively large percent of the medicament reaches the infant's eye region; and,
(d) the mask dead space is larger than necessary thus the dose of medication inhaled by the infant is suboptimal since some of the aerosol remaining in the mask at end of inspiration is lost to the environment during exhalation.

Therefore, the object of the present invention is to provide an administering apparatus that will overcome the aforementioned difficulties.

The administering apparatus 100, as provided by the present invention comprises, in a non limiting manner, the following:

(a) at least one mask 10 having at least two portions: (i) a mouth portion 11 adapted to be placed over the infant mouth. The mouth portion 11 is characterized by having at least one perforation 1. The mask will additionally comprise a nose portion 12 adapted to be placed over the infant nose. The administering apparatus 100 additionally comprises at least one spacer 20 at least partially reversibly coupled to said nose portion; (c) calming or soothing means 30 at least partially reversibly coupled to said mouth portion, adapted to be sucked by said infant such that said infant remains calm and readily accepts the mask; (d) at least one container 40 reversibly coupled to a spacer or, more efficiently, a one-way valved aerosol holding chamber or reservoir 20, adapted to contain the medicament to be administered to the infant during normal breathing; the infant is provided with the medicament by complete coordination between the sucking of the calming means and inhalation of the medicament through the nose portion 12.

The mask 10 additionally comprises an adaptor 80 reversibly coupled to the nose portion of the mask and having means to prevent the medicament from being exhaled in the direction of the infant's eyes.

The mask 10 is especially design and constructed so as to provide a 80-100% hermetic sealing between same and said infant mouth area and nose area when fitted and whilst the infant sucks said calming or feeding means.

According to one embodiment of the present invention, the 80-100% hermetic sealing will be enabled due to an accordion configuration on the contours of the mask wall 10. Reference is now made to FIGS. 2a and 2b illustrating mask 10 prior to and after the suction of the soothing or feeding means by the infant. FIG. 2a illustrates mask 10 prior to the suction and FIG. 2b illustrates the mask after the suction was applied. As can be seen from the figures, due to the accordion design and configuration of the mask 10, when the infant sucks the soothing or feeding means, the volume of the mask is substantially reduced so as 80-100% hermetic sealing is obtained and the mask dead space is minimized.

Once the infant will be presented with the soothing or feeding means, the 80-100% hermetic sealing will be enabled due to the suction of said relaxing or feeding means by said infant.

It should be pointed out that the 80-100% hermetic sealing will also be enabled due the materials from which the mask will be made of The materials should be highly flexible such as silicone of an appropriate thickness or durometer.

According to another embodiment of the present invention, mask 10, when fitted, provides an internal negative pressure higher than about 5 mbar and lower than about 10 mbar, for a period of time t that is obtained while the infant sucks said soothing/calming or feeding means.

Furthermore, the amount of time t needed to empty out said spacer of said medicament normalized by the physical volume of said spacer is reduced by at least 25% as measured according to different labeling methods. Furthermore, the amount M of said medicament that accumulates on the walls of the spacer is reduced by at least 25% with respect to conventional masks.

The different labeling methods can be selected from a group consisting of radio labeling methods, different marking methods, chemical labeling methods or any combination thereof.

Moreover, the effective amount C of said medicament administered to said infant is at least 1.25 times greater than the standard administering devices as measured by labeling methods.

According to another embodiment of the present invention, the labeling methods are selected from a group selected from radio labeling methods, different marking methods, chemical labeling methods or any combination thereof.

According to yet another embodiment of the present invention, the mask is coated and/or embedded with flavor/smell enhancement material.

The relaxing or feeding means are selected from a group consisting of pacifier or bottle already familiar to the infant.

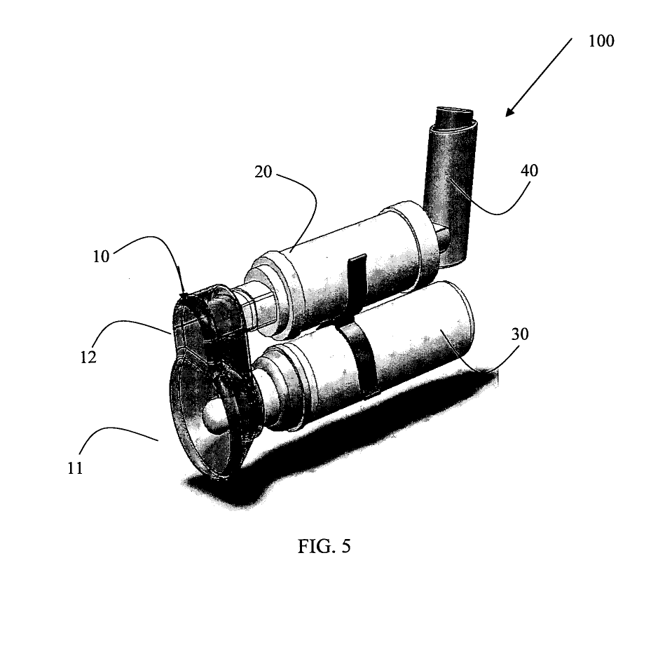

In FIG. 4 the relaxing means 30 is a pacifier and in FIG. 5 the feeding or relaxing means 30 is a bottle.

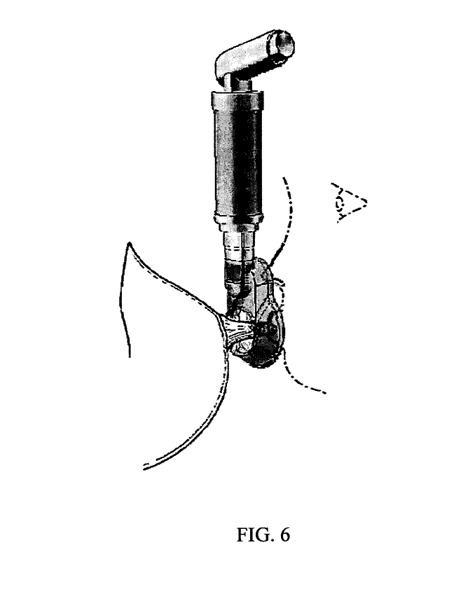

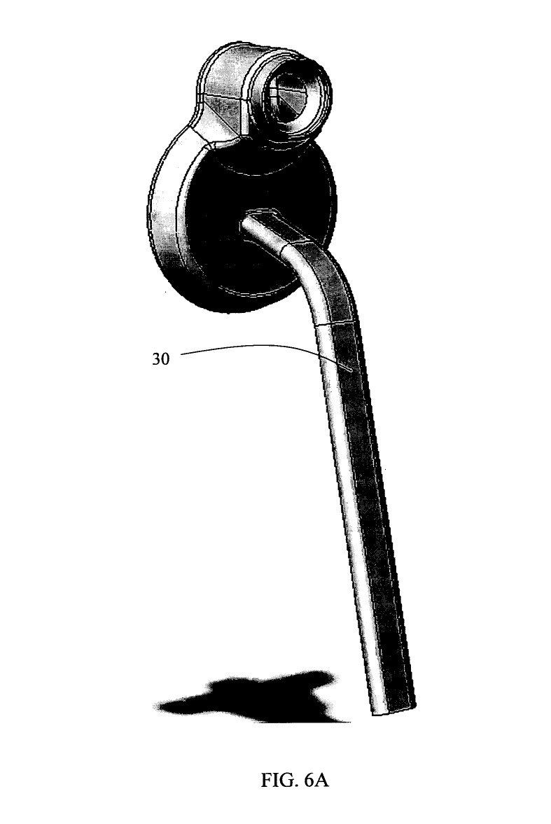

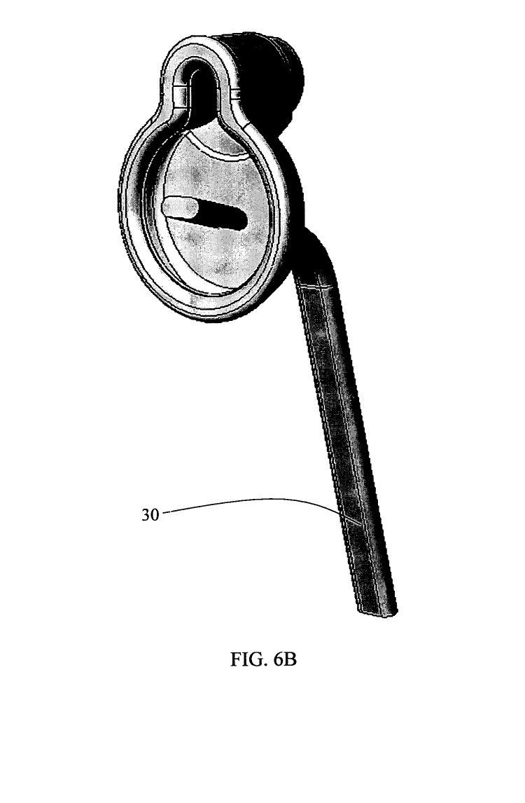

Reference is now made to FIG. 6, illustrating another preferred embodiment of the administering apparatus 100. As can be seen from the figure, the mouth-nose mask within the administering apparatus 100 is coupled to a woman's breast nipple 70.

As mentioned above, one of the advantages of the present invention is the ability to couple the mouth portion 11 within the mouth-nose mask 10 to a woman's nipple. i.e., using a woman's breast nipple as the soothing and feeding means. Again, the great advantage of using a woman's breast nipple is the fact that the breast-feeding process, in general, and a woman's nipple, in particular, is one of the most relaxing activities for the infant. Therefore, combining the breast-feeding process or the woman's breast nipple within a medicament administering device (i.e., coupling the mask to the nipple) may be highly advantageous. According to said embodiment, the mouth portion is adapted to be reversibly coupled to the contour of a woman's breast and said perforation is adapted to be reversibly coupled to the woman's nipple.

Reference is lastly made to FIGS. 6a and 6b, illustrating another preferred embodiment of the present invention according to which the mask is coupled to feeding means 30 (e.g., straw).

According to another embodiment of the present invention, the proportion of infants accepting the mouth-nose mask is more than about 50% according to the VT acceptance scale.

According to another embodiment of the present invention, the amount of time the infant remains calm and soothed is at least 20 sec, more preferably several minutes according to VTT scale.

According to another embodiment of the present invention, the amount of time the infant remains calm and soothed is increased by one stage of the BA scale

The administering apparatus 100 operates as follows:

First an administering apparatus as described above is provided. Subsequently, a spacer or more preferably a valved aerosol holding chamber/reservoir 20 is reversibly coupled to the nose portion 11. Next, the container 40 is reversibly coupled to the spacer 20 and the relaxing or feeding means are coupled to the perforation 1 in the mouth portion. Then, the mouth portion 11 of mask 10 is gently placed over the infant's mouth and the nose portion 11 is placed over the infant's nose. Then when the infant sucks on the soothing or feeding means, a 80-100% hermetic seal between the mask and the infant's mouth area and nose area is achieved due to the configuration and design of the mask. The aerosol medicament is thus efficiently administered to the infant by the complete coordination between sucking the calming/soothing means 30 and inhaling the medicament.

Once the administering apparatus 100, namely the mask 10, is gently placed over the infant's mouth and nose, the infant is efficiently provided with the medicament by the complete coordination between sucking of the soothing means and inhalation of the medicament through the nose portion of the mask 10.

Furthermore, when using the administering apparatus 100, namely the mask 10, improvement in the delivery of the medication to the infant from the valved aerosol reservoir is obtained as a result of the one-way pumping action provided by the in relatively marked in and out movement of the front wall of the mask in the direction of and away from the infant's face.

The major improvements of the medicament administration apparatus 100, namely the mask 10 are as follows:

Due to the special construction and design of the mask, a 80-100% sealed environment is created so as (i) the amount of time t in which said medicament remains within the spacer is reduced by at least 25% as measured by different labeling methods, so that the amount M of the medicament that accumulates on the walls of the spacer is reduced by at least 25% with respect to conventional masks (such as those listed in table 1).

According to some embodiments the infant is first accustomed to the mask by placing said mask (coupled to the calming means—e.g., the pacifier) over the face of the infant without any medicament being administered. Once the infant is apparently completely familiar and comfortable with the mask the treatment can begin.

According to another embodiment of the present invention, the medicament given to the infants can be aroma-therapeutic medicaments.

According to another embodiment the medicament given to the infants is oxygen.

According to another embodiment the medicament given to the infants is anesthetic gas (e.g., ethers. halogenated ethers, desflurane (2,2,2-trifluoro-1-fluoroethyl-difluoromethyl ether), sevoflurane (2,2,2-trifluoro-1-[trifluoromethyl]ethyl fluoromethyl ether), and isoflurane (2-chloro-2-(difluoromethoxy)-1,1,1-trifluoro-ethane)) for use as local anesthesia, different regional anesthesia, general anesthesia.

The various labeling methods can be selected from a group consisting of radio labeling methods, different marking methods, chemical labeling methods or any combination thereof.

It should be pointed out that the 80-100% hermetic sealing will also be facilitated by the design of the mask and the materials from which it is made. The materials should be highly flexible such as silicone.

A further improvement is the fact that the effective amount C of the medicament administered to the infant is at least 1.25 times greater than that administered by standard, currently available, devices as measured by radio labeling methods (such as those described in table 1).

According to yet another embodiment of the present invention, the mask within said administering apparatus 100 is coated and/or embedded with flavour/smell enhancement material.

It is acknowledged that aerosol reservoir 20 is provided with a unidirectional valve enabling the entrance of medicament to the nose portion 10 during inhalation whilst preventing the entrance of exhaled air back to the reservoir device 20.

According to another embodiment of the present invention, a better medicament particle distribution is obtained when using the device as described above, such that the proportion of particles having a particles size smaller than 5 micrometers is increased by at least 25%.

According to another embodiment of the present invention, the proportion of infants accepting the mouth-nose mask is more than about 50% according to the VT acceptance scale.

According to another embodiment of the present invention, the amount of time the infant remains relaxed and soothed is at least 20 sec, more preferably several minutes according to VTT scale.

According to another embodiment of the present invention, the amount of time the infant remains calm and soothed is increased by one stage of the BA scale

It should be emphasized that the mask will be made from silicone or any other highly flexible material.

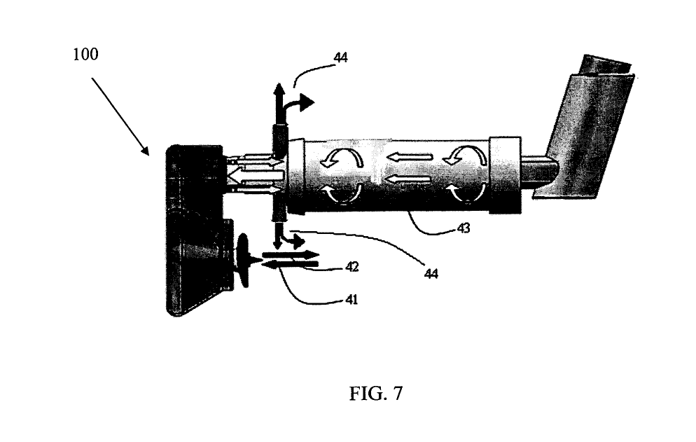

Reference is now made to FIG. 7, illustrating the medicament flow within the administering apparatus 100 which is fully coordinated with the movement of the soothing means. Arrow 41 describes the movement of the soothing means 30 due to the suction applied regularly and intermittently by the infant. Arrow 42 describes the movement of the soothing means 30 once suction intermittently ceases.

Arrows 43 describes the medicament aerosol motion within the administering apparatus 100 and Arrows 44 illustrate the direction taken by the exhaled air.

Reference is now made to FIG. 8 illustrating the dedicated adaptor, 80 coupled to the nose portion 12 of the mouth-nose mask 10. The adaptor enables a universal connection to a spacer, aerosol powder inhaler, dry powder delivery systems, nebulizer or any medicament delivery source.

The adaptor 80 preferably comprises a membrane 81 and at least one aperture (namely, exit passage) 85 for enabling exhaled medicament to exit the mouth-nose mask 10 in a predetermined direction (namely, away from the eye region).

It is emphasized that the exit passages 85 are designed and constructed in such a manner that said predetermined direction of the exhaled medicament exiting the nose portion of the mask is directed away from the infant's face and thus little or none of the exhaled aerosol would be expected to reach the infant's eye region.

Inhaled air (see arrow 82) entering the mask 10 through the inhalation tube 83 is inhaled by the infant. The exhaled air (containing also some of the medicament) is exhaled in direction 84 through said at least one aperture exit passage 85.

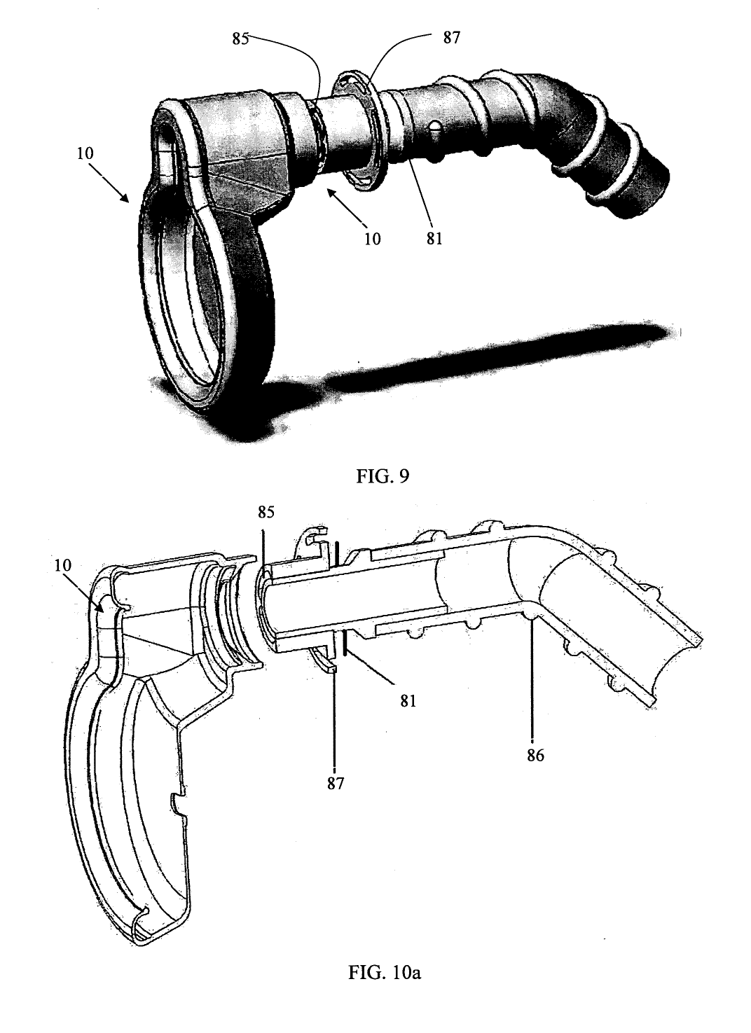

Reference is now made to FIGS. 9-10b illustrating different prospective view and a cross sectional view of nose-mouth mask 10 coupled to the adaptor 80.

As mentioned above, one of the main advantages of the adaptor according to the present invention is the ability to distant the exhaled air from the user's eyes region.

The figures illustrate the apertures (namely, the exit passage) 85, from which the exhaled air containing the medicament exits.

According to one embodiment, the adaptor additionally comprises a membrane 81 for filtering the inhaled air/exhaled air (see FIG. 9).

According to one embodiment, the adaptor additionally comprises perforated disc 87 for coupling the adaptor to a cap-like retainer to be fitted upon/around the users head.

In FIGS. 9-10 the adaptor is coupled to a flexible tube 86. Said flexible tube is for coupling with the drug container that is characterized by a small volume wet nebulizer.

As mentioned above, the adaptor is a universal adaptor that can be coupled to spacer, aerosol powder inhaler, dry powder delivery systems, nebulizer or any medicament delivery source.

FIG. 10b provides a closer view of the adaptor 80. Again, the apertures (namely, the exit passage) 85 are illustrates as well as the perforated disc 87 for coupling the adaptor to a cap-like retainer to be fitted upon/around the users head.

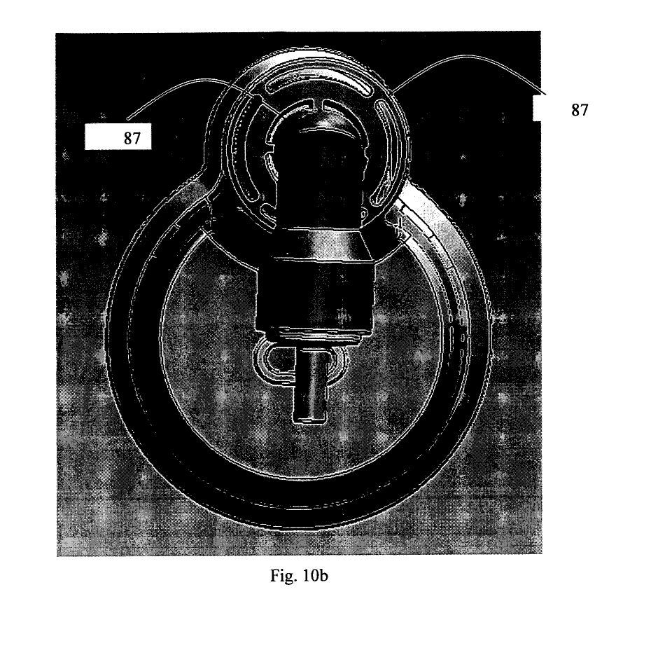

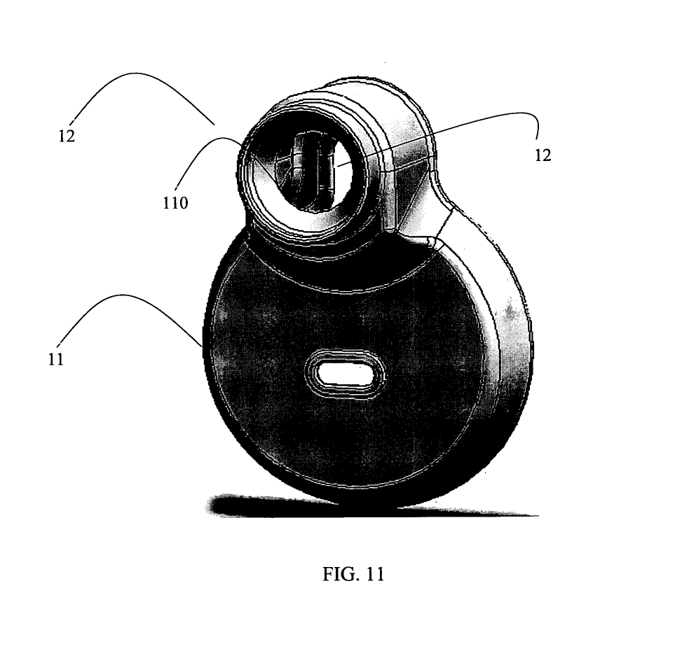

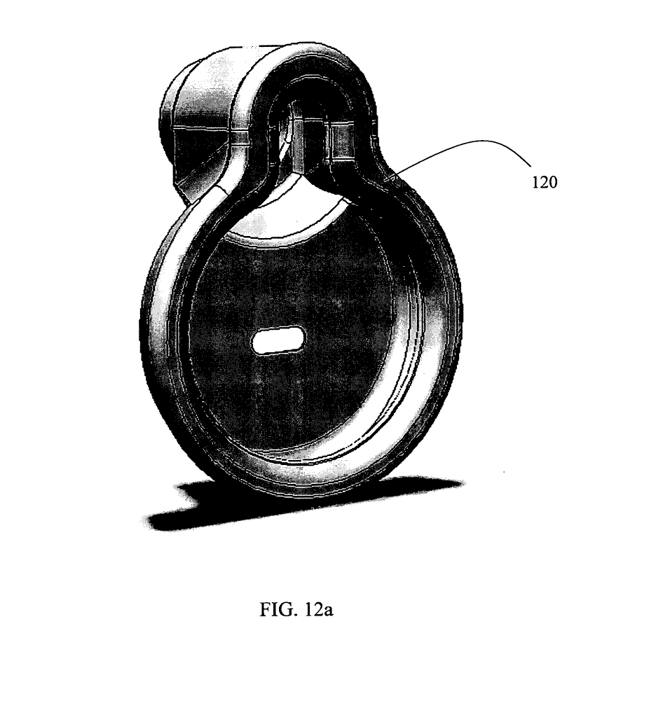

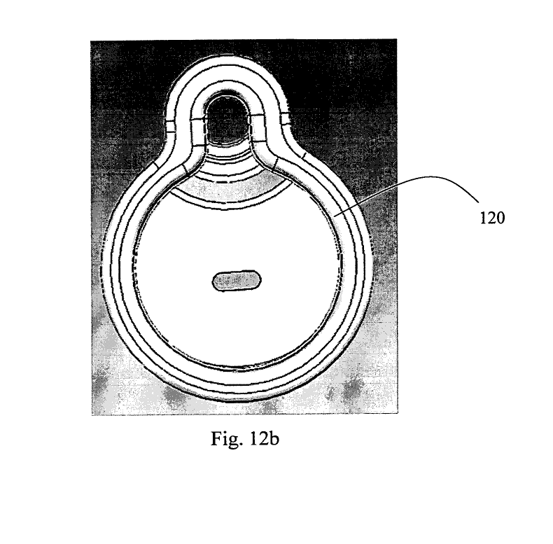

Reference is now made to FIGS. 11-12b which illustrate different views of the mask 10. As previously described, the mask 10 comprises a nose portion 12 and a mouth portion 11.

According to another embodiment of the present invention, the nose portion is characterized by a back side, which eventually be place upon the nose and a front side, which will eventually be coupled to the adaptor or the spacer, aerosol powder inhaler, dry powder delivery systems, nebulizer or any medicament delivery source.

According to said embodiment said front face of said nose portion comprise a rim 110, adapted to be internally folded (internally clasped).

Said rim 110 enables the firm coupling of said adaptor 80.

FIGS. 12a and 12b illustrates an important feature provide by the nose-mouth mask of the present invention which is the frame or edge 120 surrounding the back/-internal side/surface of the mask (or in other words, a rim).

The rim 120 has several advantages, among other, better sealing. Said advantage is obtained simply by increasing the cross section of the rim which comes into contact with the user's face.

The rim 120 also enables the mask to fit to a wide range of facial cross sections.

In other words, the mask will fit to an infant of several months and will accompany him till he will be of several years of age, especially from the age of a month to the age of 28 months.

As described above, the mask is configured such that the ‘Dead-Space’ is minimized, yet still medication can reach the infant's mouth.

Thus, even when an infant has a cold—he will be able to breath through the mouth.

The same is enabled by providing the mouth portion with dimensions such that the volume of the same is minimized and the following ratio is being held true:

Vₙₒₛₑ>>a*Vₘₒᵤₜₕ

where Vₙₒₛₑ is the volume of the nose portion; Vₘₒᵤₜₕ is the volume of the mouth portion; and a is likely to be in the range of 1.5 to 2.

Reference is now made to FIG. 13 illustrating another embodiment of the present invention, in which the nose-mouth mask 10 comprises means (e.g., protrusion 130) adapted to enable a firm grip of the mask (prevent of slipperiness).

According to said embodiment, at least a portion of the mask's external surface comprises a plurality of bulges (e.g., protrusion), 130 so as to provide a rough surface.

It is still an object of the present invention to provide a universal adaptor adapted to be coupled to a nose-mouth mask and deliver medicament to an infant, comprising (a) at least one nose end, adapted to be reversibly coupled to said nose portion of a nose-mouth mask; and, (b) at least one MDS end, adapted to be reversibly coupled to at least one MDS; wherein said at least one nose end comprising at least one aperture for enabling an exhaled medicament to exit said nose portion of said nose-mouth mask in a predetermined direction. It is still an object of the present invention to provide the universal adaptor as defined above, wherein said exhaled medicament exits said mouth-nose mask in said predetermined direction substantially without reaching said infant's eye-region.

### EXAMPLES

Examples are given in order to prove the embodiments claimed in the present invention. The example, which is a clinical test, describes the manner and process of the present invention and set forth the best mode contemplated by the inventors for carrying out the invention, but are not to be construed as limiting the invention.

### Example 1

### Dead Space Volume Testing Report

The following test was performed in order to evaluate the dead space of several preferable nose-mouth masks.

The aim of the following testing was to compare different full face masks so as to obtain the dead space volume.

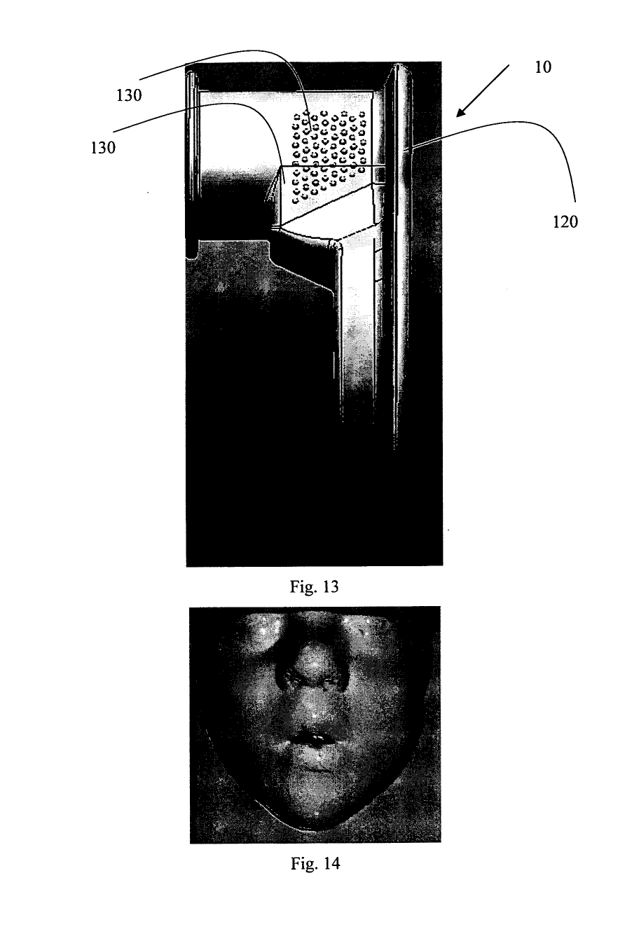

During the tests, a model of a 22 months toddler's face (obtained from a computed axial tomography CT scan and 3-D printing technology) was used. FIG. 14 illustrates the 22 months toddler's face model used.

The following masks were used:

Easyhale™, Trudel™, Funhale™, NebuChamber™, Respironics, Voyage by CTS.

The following tests were performed:

The masks were attached onto the model face with special glue, in order to avoid leakages at the areas whereupon the masks were in contact with the model (i.e., the face). Thereafter, liquid was injected, through the mask, which filled the entire space.

Once there were no air bubbles, within the mask, the liquid was drawn into a measuring cup.

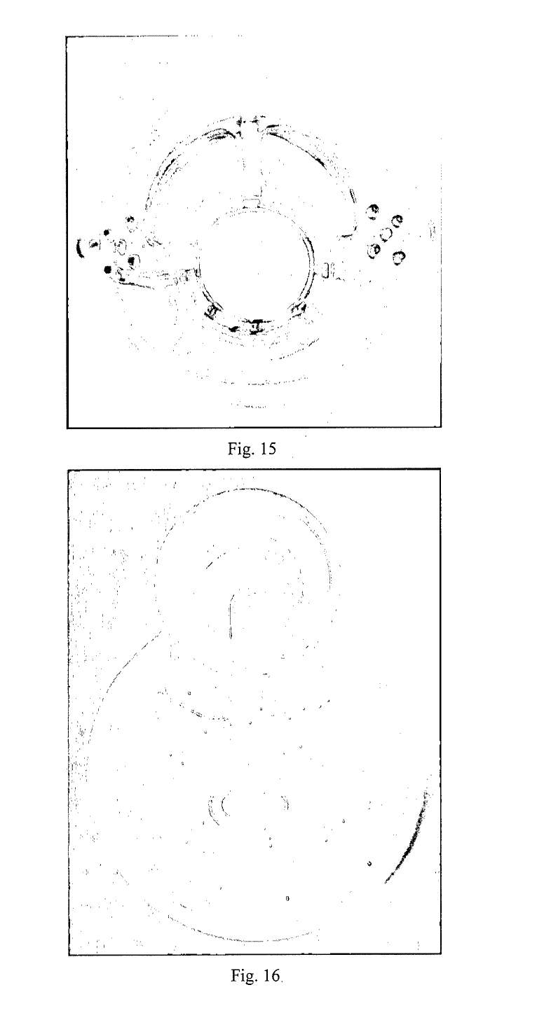

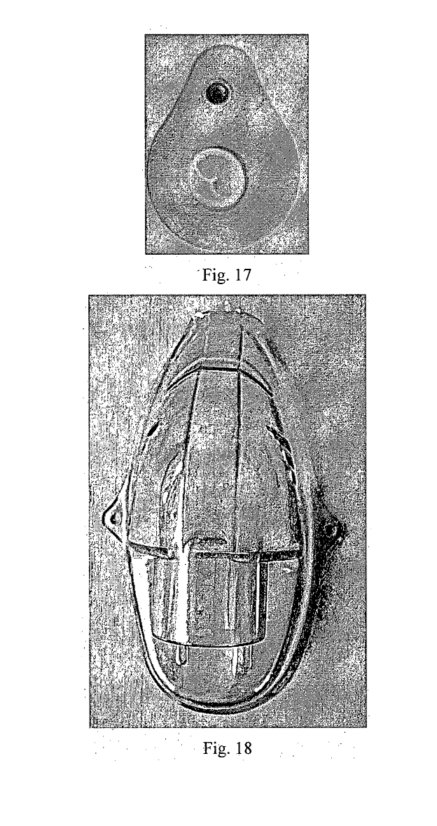

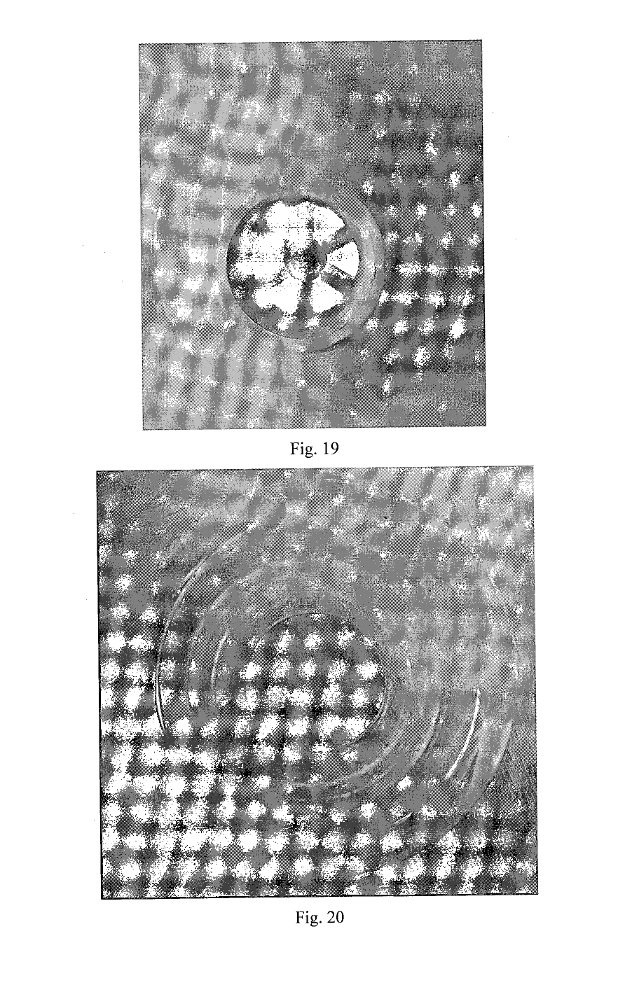

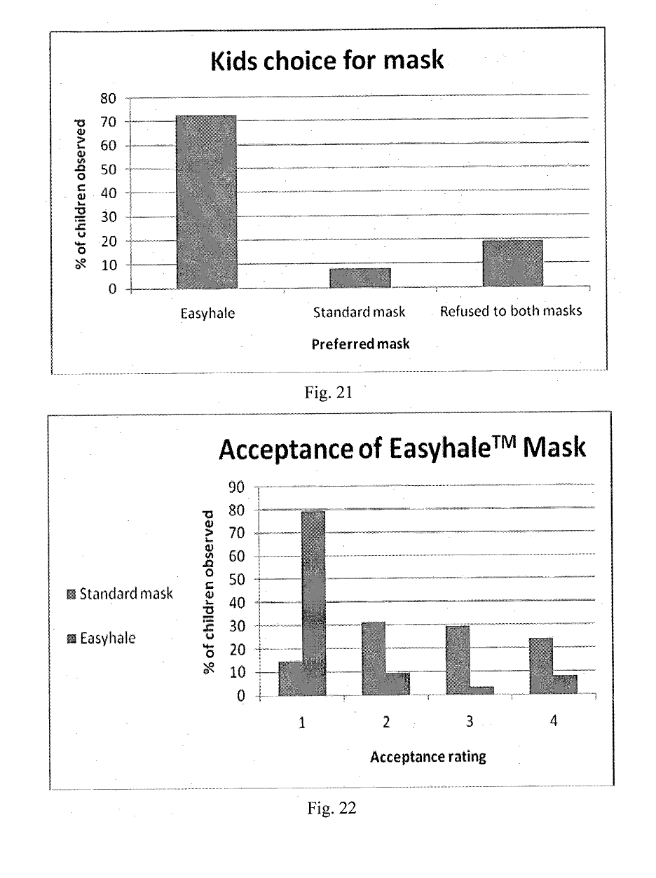

All the testing were re-performed three times with each mask and the following averages results were obtained.

| Name of manufacturer | Dead space volume |
|---|---|
| Respironics See FIG. 15 | 75 cc |
| The mask of the present | 28 cc |
| invention, Easyhale ™, see | |
| FIG. 16 | |
| Trudel ™, see FIG. 17 | 18 cc |
| Standard mask in Israel, | 35 cc |
| Voyage by CTS | |
| see FIG. 18 | |
| Funhale ™, see FIG. 19 | 55 cc |
| NebuChamber ™, see | 41 cc |
| FIG. 20 | |

### Example 2

### Evaluation of the Mask as Provided by the Present Invention, Easyhale™, Mask Acceptance by Children—A Field Observations Trial

An observation trial was designed in order to evaluate the acceptance of the Easyhale™ mask by the target users, when compared to standard inhalation mask that presents the “regular” treatment in today's market.

60 kids of ages 1-4 where exposed to Easyhale™ mask with the pacifier application and to a standard inhalation mask, namely Trudel™ and Voyage by CTS.

Kids were asked to choose their preferable mask among the two suggested masks, for their potential inhalation treatment.

Reference is now made to figure which illustrates a significant preference for the Easyhale™ mask.

Approx. 70% of the kids preferred the mask as provided by the present invention.

Acceptance of the Easyhale™ mask with the pacifier application was compared to the standard mask (Trudel™ and Voyage by CTS) by using a pre-defined rating scale as follows:
1—Easily accepted
2—Accepted with some resistance
3—Kid Expressed anxiety
4—Kid refused to accept

Reference is now made to FIG. 22 which illustrates the acceptance of the mask by the observed kids.

As can be seen almost 80% of the kids easily accepted the mask of the present invention. 10% of the kids accepted the mask of the present invention with some resistance.

Less than 10% of the kids expressed anxiety when taking the mask of the present invention with some resistance.

Less than 10% of the kids refused to take the mask of the present invention with some resistance.

The acceptance of Easyhale™ mask with a straw application was evaluated on kids ranging from age 2-4 and compared to a standard inhalation mask (Trudel™ and Voyage by CTS).

24 kids were asked to choose their preferred mask for their potential treatment, where the two masks of choice were the Easyhale™ mask with a straw and matching adaptor and a standard inhalation mask.

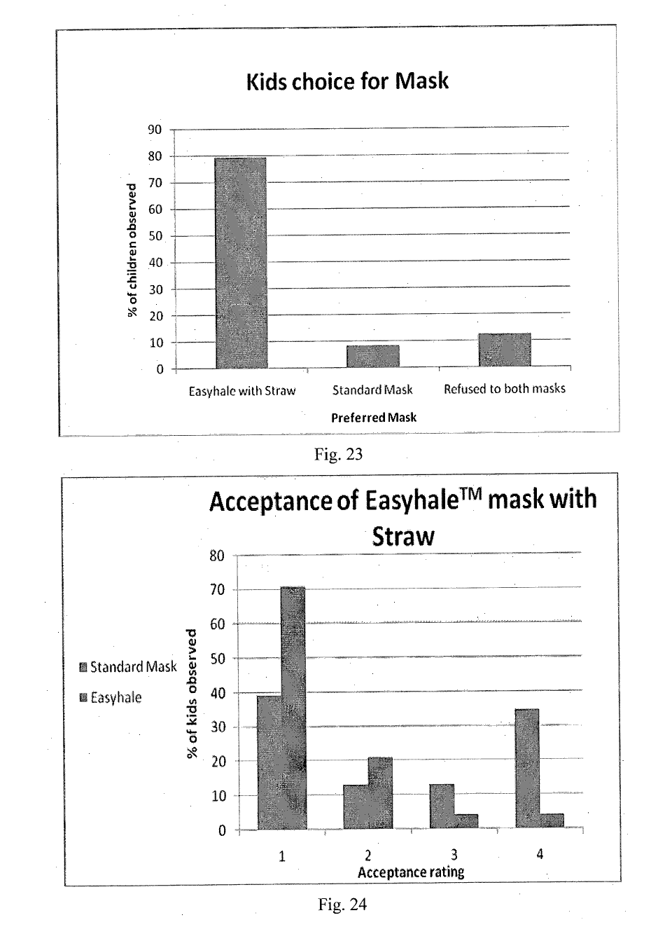

Here again, a significant preference for the Easyhale™ mask was observed as summarized in FIG. 23.

Acceptance was rated also for the use of Easyhale™ with a straw and compared to the acceptance rates of the standard inhalation mask.

As in the case of the Easyhale™ mask with the pacifier, kids' were fond with the mask-straw combination and were willing to use it (see FIG. 24).

### Conclusions:

The observation study strongly support the preference of kids to the Easyhale™ mask on top of the standard mask that is in use today.

The acceptance rate for the Easyhale™ mask, absolutely and when compared to the standard masks in market, reflects the high compliance of the device that would potentially result in reducing child's anxiety related to inhalation treatment, and will minimize omitting treatments as a result of child's rejection of mask and treatment.

### Example 3

### The Ratio Between the Mouth Volume, V_{Mouth} and Nose Volume V_{Nose}

The following tests were performed in order to evaluate the Vmouth and Vnose.

The mask of the present invention was divided into the nose portion and the mouth portion.

Then, stuffing was introduced and filled the nose portion with liquid in order to evaluate the volume.

The above was reproduced 3 times.

The nose volume was calculated to in the range of 17-18 cc, the mouth portion was calculated to be 10 cc.

Thus, the ratio Vₙₒₛₑ/Vₘₒᵤₜₕ is likely to be in the range of 1.5 to 2.

In the foregoing description, embodiments of the invention, including preferred embodiments, have been presented for the purpose of illustration and description. They are not intended to be exhaustive or to limit the invention to the precise form disclosed. Obvious modifications or variations are possible in light of the above teachings. The present embodiments were chosen and described to provide the best illustration of the principles of the invention and its practical applications, and to enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

1-116. (canceled)

117. An infant mouth-nose mask for administering medicaments to infants, comprising:
a. a mouth portion, defining a confined mouth volume, placed over the mouth of said infant characterized by having at least one perforation reversibly coupled to calming or feeding means such that said infant remains relaxed and soothed while sucking said calming or feeding means; said confined mouth volume is characterized by volume, Vₘₒᵤₜₕ; and,
b. a nose portion, defining a confined mouth volume, placed over the nose of said infant; said nose portion is adapted to be in fluid connection with a medicament delivery source (MDS); said confined nose volume is characterized by volume, Vₙₒₛₑ;
wherein said Vₙₒₛₑ>>a*Vₘₒᵤₜₕ such that the dead-space of said mask is not greater than 28 cc, and a is in the range of about 1.5 to about 2.

118. The infant mouth-nose mask according to claim 117, wherein said mouth-nose mask is characterized by an external surface and an internal surface; said internal surface is adapted to come into contact with said infant's face; further wherein said internal surface comprising a frame or edge surrounding the same; said frame is adapted to (a) increase the cross section area which come into contact with said infant's face; and, (b) to enable the adjustment of said mouth-nose mask to a wide range of infant's age and infant's facial cross sections.

119. The infant mouth-nose mask according to claim 117, wherein said medicament is in the form of aerosol, gas powder or any combination thereof; further wherein said medicament is anesthetic gas Asthma medicaments, corticosteroids, Leukotriene antagonists, beta-agonists; said anesthetic gas is selected from ethers, halogenated ethers, desflurane (2,2,2-trifluoro-1-fluoroethyl-difluoromethyl ether), sevoflurane (2,2,2-trifluoro-1-[trifluoromethyl]ethyl fluoromethyl ether), and isoflurane (2-chloro-2-(difluoromethoxy)-1,1,1-trifluoro-ethane) and any combination thereof, for use as local anesthesia, different regional anesthesia, general anesthesia.

120. The infant mouth-nose mask according to claim 117, wherein at least one of the following is being held true (a) said mask is provided for physiotherapy breathing, pulmonary rehabilitation program, acute or chronic pulmonary disorders or any combination thereof; (b) at least a portion of said mask's external surface comprises a plurality of bulges so as to provide a rough surface; (c) at least a portion of said mask's external surface is coarsed.

121. The infant mouth-nose according to claim 117, additionally comprising at least one adaptor reversibly coupled to said nose portion, comprising at least one aperture for enabling exhaled medicament to exit said mouth-nose mask in a predetermined direction; further wherein said exhaled medicament exits said mouth-nose mask by means of said at least one aperture in said predetermined direction substantially without reaching said infant's eye region.

122. The infant mouth-nose according to claim 121, wherein said adaptor is integrated within said nose portion of said mask.

123. The infant mouth-nose according to claim 117, wherein nose portion is characterized by a back side, adapted to be placed upon said nose of said infant and a front side; said front said is at least partially reversibly coupled to a spacer, aerosol powder inhaler, dry powder delivery systems, nebulizer or any medicament delivery source (MDS).

124. The infant mouth-nose according to claim 123, wherein said front face of said nose portion comprise an internally folded rim; further wherein said internally folded rim is adapted to enable reversible coupling of at least one selected from a group consisting of a spacer, aerosol powder inhaler, dry powder delivery systems, nebulizer or any medicament delivery source (MDS) to said nose portion.

125. The infant mouth-nose according to claim 121, wherein said adaptor comprises a perforated disc for coupling said adaptor to a cap-like retainer to be fitted upon or around the users head.

126. An infant mouth-nose mask for administering medicaments to infants while substantially preventing said medicament from reaching the eye region of said infants, comprising:
a. a mouth portion, defining a confined mouth volume, placed over the mouth of said infant characterized by having at least one perforation reversibly coupled to calming or feeding means such that said infant remains relaxed and soothed while sucking said calming or feeding means; said confined mouth volume is characterized by volume, Vₘₒᵤₜₕ; and,
b. a nose portion, defining a confined mouth volume, placed over the nose of said infant; said nose portion is adapted to be in fluid connection with a medicament delivery source (MDS); said confined nose volume is characterized by volume, Vₙₒₛₑ;
c. at least one adaptor reversibly coupled to said nose portion, comprising at least one aperture for enabling exhaled medicament to exit said mouth-nose mask in a predetermined direction;
wherein said exhaled medicament exits said mouth-nose mask by means of said aperture in said predetermined direction substantially without reaching said infant's eye region; further wherein said Vₙₒₛₑ>>a*Vₘₒᵤₜₕ such that the dead-space of said mask is not greater than 28 cc, and a is in the range of about 1.5 to about 2.

127. The infant mouth-nose mask according to claim 126, wherein at least one of the following is being held true (a) said adaptor is integrated within said nose portion of said mask; (b) said mask when fitted is adapted to provide a 80-100% hermetic sealing between said mask and said mouth and nose of said infant whilst said infant sucks said calming or feeding means; (c) at least a portion of the contours of the side-walls of said mask is accordion-like shaped so as to provide said 80-100% hermetic sealing and at the same time minimizing said mask's dead space.

128. The infant mouth-nose mask according to claim 126, wherein at least one of the following is being held true (a) said medicament is in the form of aerosol, gas powder or any combination thereof; (b) said medicament is selected from Asthma medicaments, corticosteroids, Leukotriene antagonists, beta-agonists or any combination thereof; (c) said medicament is anesthetic gas selected from ethers. halogenated ethers, desflurane (2,2,2-trifluoro-1-fluoroethyl-difluoromethyl ether), sevoflurane (2,2,2-trifluoro-1-[trifluoromethyl]ethyl fluoromethyl ether), and isoflurane (2-chloro-2-(difluoromethoxy)-1,1,1-trifluoro-ethane) and any combination thereof, for use as local anesthesia, different regional anesthesia, general anesthesia or any combination thereof.

129. The infant mouth-nose mask according to claim 126, wherein at least one of the following is being held true (a) said mask is provided for physiotherapy breathing, pulmonary rehabilitation program, acute or chronic pulmonary disorders or any combination thereof; (b) said calming or feeding means are selected from a group consisting of pacifier, straw, sippy attachment or a bottle's nipple already familiar to said infant; (c) said pacifier is integrated within said mouth portion of said mask; (d) said mask additionally comprises flavor or smell enhancement material embedded within and/or coating said mask; (e) said calming or feeding means is a woman's breast nipple such that said mouth portion is adapted to be reversibly coupled to the contour of a woman's breast and said perforation is adapted to be reversibly coupled to said woman's nipple; (f) at least a portion of said mask's external surface comprises a plurality of bulges so as to provide a rough surface; (g) at least a portion of said mask's external surface is coarsed; and any combination thereof.

130. The infant mouth-nose mask according to claim 126, wherein said mask when fitted provides an internal negative pressure higher than about 5 mbar and lower than about 10 mbar, for a period of time t, whilst said infant sucks said calming or feeding means such that
a. a 80-100% hermetic sealing between said mask and said infant mouth and nose during said period of time t is obtained;
b. the amount of time t needed to empty out said spacer from said medicament normalized by the physical volume of said spacer is reduced by at least 30%; so that the amount M of said medicament that accumulates on the walls of said spacer is reduced by at least 20% with respect to conventional masks; and,
c. the effective amount C of said medicament administered to said infant is at least 1.25 times greater than standard, currently available devices as measured by labeling methods; wherein said labeling method is selected from a group consisting of radio labeling methods, various marking methods, chemical labeling methods or any combination thereof.

131. The infant mouth-nose mask according to claim 126, wherein said mouth-nose mask is characterized by an external surface and an internal surface; said internal surface is adapted to come into contact with said infant's face; further wherein said internal surface comprising a frame or edge surrounding the same; further wherein said frame is adapted to (a) increase the cross section area which come into contact with said infant's face; (b) enable the adjustment of said mouth-nose mask to a wide range of infant's ages and infant's facial cross sections.

132. The infant mouth-nose according to claim 126, wherein at least one of the following is being held true (a) said nose portion is characterized by a back side, adapted to be placed upon said nose of said infant and a front side; (b) said front said is at least partially reversibly coupled to a spacer, aerosol powder inhaler, anesthetic drugs as liquid, aerosols, or gas, dry powder delivery systems, nebulizer or any medicament delivery source (MDS); (c) said front face of said nose portion comprise an internally folded rim; (d) said internally folded rim is adapted to enable reversible coupling of at least one selected from a group consisting of a spacer, aerosol powder inhaler, dry powder delivery systems, nebulizer or any medicament delivery source (MDS) to said nose portion; (e) said adaptor comprises a perforated disc for coupling said adaptor to a cap-like retainer to be fitted upon or around the users head; and any combination thereof.

133. The infant mouth-nose according to claim 126, additionally comprising at least one container in fluid connection with said MDS, containing aerosol medicament to be administered to said infant

134. The infant mouth-nose according to claim 126, wherein said MDS is selected from, valved aerosol holding chamber/reservoir device, wet nebulizer, aerosol powder inhaler, anesthetic drugs as liquid, aerosols, or gas, dry powder delivery systems or any combination thereof.

135. A method for administering medicaments to infants while substantially preventing said medicament from reaching the eye region of said infants, comprising steps of:
a. providing an administering apparatus, consisting of:
i. a mouth-nose mask having (i) a mouth portion, defining a confined mouth volume, placed over the mouth of said infant; said mouth portion comprising at least one perforation reversibly coupled to calming or feeding means such that said infant remains soothed whilst sucking said calming or feeding means; (ii) a nose portion, defining a confined nose volume, placed over the nose of said infant; and, (iii) at least one adaptor comprising at least one aperture for enabling exhaled medicament to exit said mouth-nose mask in a predetermined direction;
ii. at least one medicament delivery source (MDS), in fluid connection with said nose portion;
iii. calming or feeding means at least reversibly mounted via said mouth portion into said infant's mouth; adapted to be sucked by said infant such that said infant remains soothed while sucking said calming or feeding means; and,
iv. at least one container in fluid connection with said MDS, containing a medicament to be administered to said infant;
b. reversibly coupling said relaxing or feeding means to said perforation within said mouth portion;
c. gently placing said mouth portion over the infant's mouth so as to surround said mouth of said infant and define a confined mouth volume;
d. gently placing said nose portion over the infant noise so as to surround said nose of said infant and define a confined n volume;
e. reversibly coupling said MDS to said nose portion;
f. reversibly coupling said at least one container to said MDS;
g. efficiently administering said medicament to said infant by enabling said infant to suck said calming or feeding means while fully coordinating breathing said medicament; and,
h. directing the exhaled medicament from said mouth-nose mask in said predetermined direction by means of said at least one aperture, thereby substantially preventing said exhaled medicament from reaching said eye region of said infant.

136. The method for efficiently administering a medicament to an infant according to claim 135, wherein at least one of the following is being held true (a) said steps of d and c are performed simultaneously; (b) said step d is preformed prior to said step c; (c) said step (f) of efficiently administering said medicament is performed such that a 80-100% hermetic sealing between said mask and said infant mouth and nose is provided; and any combination thereof.

137. The method for efficiently administering a medicament to an infant according to claim 135, wherein at least one of the following is being held true (a) said adaptor is integrated within said nose portion of said mask; (b) said pacifier is integrated within said mouth portion of said mask; (c) said confined nose volume is characterized by volume, Vₙₒₛₑ; said confined mouth volume is characterized by volume, Vₘₒᵤₜₕ; such that said Vₙₒₛₑ>>a*Vₘₒᵤₜₕ such that the dead-space of said mask is not greater than 28 cc, and a is in the range of about 1.5 to about 2; (d) said mouth-nose mask is characterized by an external surface and an internal surface; said internal surface is adapted to come into contact with said infant's face; (e) said internal surface comprising a frame or edge surrounding the same; said frame is adapted to (a) increase the cross section area which come into contact with said infant's face; (b) enable the adjustment of said mouth-nose mask to a wide range of infant's age and infant's facial cross sections and any combination thereof.

138. The method for efficiently administering a medicament to an infant according to claim 135, additionally comprising at least one step selected from a group consisting of (a) selecting said calming or feeding means from a group consisting of pacifier, straw, sippy mouthpiece or a bottle's nipple already familiar to said infant; (b) coating and/or embedding flavor or smell enhancement material within said mask; (c) selecting said calming or feeding means from a group consisting of a woman's breast nipple; such that said mouth portion is reversibly coupled to the contour of a woman's breast and said perforation is reversibly coupled to said woman's nipple; (d) provides at least a portion of said mask's external surface with a plurality of bulges so as to provide a rough surface; (e) providing at least a portion of said mask's external surface coarsed and any combination thereof.

139. The method for efficiently administering a medicament to an infant according to claim 135, wherein at least one of the following is being held true (a) said medicament is in the form of aerosol, gas powder or any combination thereof; (b) said medicament is anesthetic gas selected from ethers. halogenated ethers, desflurane (2,2,2-trifluoro-1-fluoroethyl-difluoromethyl ether), sevoflurane (2,2,2-trifluoro-1-[trifluoromethyl]ethyl fluoromethyl ether), and isoflurane (2-chloro-2-(difluoromethoxy)-1,1,1-trifluoro-ethane) and any combination thereof, for use as local anesthesia, different regional anesthesia, general anesthesia; (c) said medicament is selected from Asthma medicaments, corticosteroids, Leukotriene antagonists, Beta-agonistor any combination thereof.

140. The method for efficiently administering a medicament to an infant according to claim 135, wherein said mask is provided for physiotherapy breathing, pulmonary rehabilitation program, acute or chronic pulmonary disorders or any combination thereof.
